(19) United States
(12) Patent Application Publication
(10) Pub. No.: US 2026/0357347 A1
(43) Pub. Date: Nov. 12, 2026

(54) MUTATION INDUCED CONFORMATIONAL CHANGES IN EPRS1 mRNA THAT PREVENT m6A METHYLATION AT DISTAL SITES

(71) Applicant: The Cleveland Clinic Foundation, Cleveland, OH (US)

(72) Inventors: Paul FOX, Cleveland, OH (US); Debjit Khan, Cleveland, OH (US); Daniel Blankenberg, Cleveland, OH (US); Fabio Cumbo, Cleveland, OH (US)

(21) Appl. No.: 19/490,618

(22) PCT Filed: Jun. 6, 2024
(86) PCT No.: PCT/US2024/032831

§ 371 Date: Dec. 5, 2025

Related U.S. Application Data
(60) Provisional application No. 63/506,401, filed on Jun. 6, 2023.
(60) Provisional application No. 63/520,261, filed on Aug. 17, 2023.

(51) Int. Cl. C12Q 1/6883

(52) U.S. Cl. CPC C12Q 1/6883; C12Q 2600/154; C12Q 2600/156; C12Q 2600/158

(57) ABSTRACT

The present invention relates to methods, kits, and compositions for testing a sample from a subject and determining: i) if said subject is A/A, A/C, or C/C at position 4444 in the Glutamyl-prolyl-tRNA synthetase 1 (EPRS1) gene or mRNA, and/or ii) if said subject expresses only the 1482T version, only the 1482P version, or both the 1482T and 1482P versions, of the EPRS1 protein; and/or iii) if said subject is m6A methylated or m6A unmethylated at A4355 and/or A4464 in said EPRS1 mRNA; and determining that the subject has hypomyelinating leukodystrophy (HLD) or other neurological condition. The present application also relates to a bioinformatic pipeline to assess if m6A-distal single-nucleotide-variations (m6Ad-SNV) affect methylation of DRACH sites.

### CROSS-REFERENCE TO RELATED APPLICATIONS

The present application claims priority to U.S. Provisional applications Ser. No. 63/520,261, filed Aug. 17, 2023, and 63/506,401, filed Jun. 6, 2023, both of which are herein incorporated by reference in their entireties.

### STATEMENT REGARDING FEDERAL FUNDING

This invention was made with government support under NS124581 and NS124547 awarded by the National Institutes of Health. The government has certain rights in the invention.

### SEQUENCE LISTING

The text of the computer readable sequence listing filed herewith, titled “CCF_42121_601_SequenceListing.xml,” created Jun. 6, 2024, having a file size of 23,523 bytes, is hereby incorporated by reference in its entirety.

### FIELD OF THE INVENTION

The present invention relates to methods, kits, and compositions for testing a sample from a subject and determining: i) if said subject is A/A, A/C, or C/C at position 4444 in the Glutamyl-prolyl-tRNA synthetase 1 (EPRS1) gene or mRNA, and/or ii) if said subject expresses only the 1482T version, only the 1482P version, or both the 1482T and 1482P versions, of the EPRS1 protein; and/or iii) if said subject is m6A methylated or m6A unmethylated at A4355 and/or A4464 in said EPRS1 mRNA; and determining that the subject has hypomyelinating leukodystrophy (HLD) or other neurological condition.

### BACKGROUND

The essential function of the 20 cytoplasmic aminoacyl-tRNA synthetases (aaRSs) is high-fidelity decoding of genetic information carried by mRNA during protein synthesis. The aaRSs catalyze ATP-dependent charging of tRNAs with cognate amino acids for delivery to the ribosome A-site. Mutations in cytoplasmic aaRSs are associated with several neurological disorders with myelination defects including epileptic encephalopathy, progressive microcephaly, Charcot-Marie-Tooth (CMT) disease, and hypomyelinating leukodystrophy (HLD) (van der Knaap and Bugiani, 2017), and there is emerging interest in aaRSs as potential therapeutics and therapeutic targets (Kwon et al., 2019). Nine of the twenty aaRS activities (in eight proteins since EPRS1 contains two covalently linked synthetase domains, glutamyl-tRNA synthetase (GluRS) and prolyl-tRNA synthetase (ProRS) reside in a cytoplasmic multi-tRNA synthetase complex (MSC) with three non-synthetase proteins, AIMP-1, -2, and -3 (Kaminska et al., 2009; Lee et al., 2004). The function of the mammalian MSC remains unclear. MSC binding to ribosomes might permit “channeling” of charged tRNAs via EF-1α into the ribosome A-site for improved translation efficiency (Barbarese et al., 1995; David et al., 2011; Kaminska et al., 2009; Negrutskii and Deutscher, 1991; Netzer et al., 2009). However, translation is not inhibited when the majority of EPRS1, or all RARS1 and QARS1, are not MSC-bound, arguing against a quantitatively significant role for the MSC in protein synthesis (Cui et al., 2021; Sampath et al., 2004).

Remarkably, pathogenic variants in seven out of eleven MSC constituents cause a broad spectrum of neurological diseases. Pathogenic variants in QARS1 and KARS1 cause progressive microcephaly (McMillan et al., 2015; Zhang et al., 2014), peripheral neuropathy (McLaughlin et al., 2010), and progressive leukoencephalopathy with brainstem and spinal cord calcifications (Itoh et al., 2019). Additionally, bi-allelic mis-sense variants in genes encoding three other aaRSs—RARS1 (Nafisinia et al., 2017), DARS1 (Taft et al., 2013), and EPRS1 (Mendes et al., 2018), cause childhood-onset HLD. Variants in two non-aaRS MSC components—AIMP1 (Feinstein et al., 2010) and AIMP2 (Shukla et al., 2018)—also cause hypomyelination, but in these cases the pathology might not result from primary hypomyelination, but rather from demyelination secondary to neurodegeneration.

The leukodystrophies are a family of more than 50 distinct heritable central nervous system (CNS) disorders characterized by diminished cerebral and cerebellar white matter due to dysregulated myelin formation or degeneration (Rutherford and Hamilton, 2019). HLD is rare, but comprises the single largest category among undiagnosed genetic leukodystrophics, which collectively impact approximately 1 in 7,500 live births, representing a major group of neurodevelopmental disorders (Elitt et al., 2018). Clinical features include severe cognitive and motor impairment appearing in early childhood or adolescence. At present there are no curative treatments; patient management includes serial brain MRI to monitor hypomyelination, genetic testing to elucidate etiology, and symptomatic treatment of neurologic and other medical complications (Pouwels et al., 2014). The causative role of multiple aaRS variants in HLD is well-established, however, a hypothesis unifying the mechanism by which variant aaRSs drive HLD has not yet emerged. For nearly all EPRS1 and RARS1 variants, the relevant aminoacyl charging activity in patient fibroblasts is reduced by about 30-50% compared to healthy controls (Mendes et al., 2020; Mendes et al., 2018). In the single study dissecting the determinants, the reduced charging activity in fibroblasts from patients with EPRS1 variants was due to reduced amount of enzyme, as well as reduced specific charging activity of recombinant protein (Mendes et al., 2018). The decrease in cellular RARS1 protein ranged from barely discernable by immunoblot to ~80% in patients with RARS1 variants (Mendes et al., 2020; Nafisinia et al., 2017). In most cases, decreased specific charging activities are attributable to variants in or near catalytic or tRNA-binding sites (Mendes et al., 2018; Taft et al., 2013). However, in none of these cases has the mechanism underlying reduced variant aaRS expression been elucidated.

### SUMMARY OF THE INVENTION

The present invention relates to methods, kits, and compositions for testing a sample from a subject and determining: i) if said subject is A/A, A/C, or C/C at position 4444 in the Glutamyl-prolyl-tRNA synthetase 1 (EPRS1) gene or mRNA, and/or ii) if said subject expresses only the 1482T version, only the 1482P version, or both the 1482T and 1482P versions, of the EPRS1 protein; and/or iii) if said subject is m6A methylated or m6A unmethylated at A4355 and/or A4464 in said EPRS1 mRNA; and determining that the subject has hypomyelinating leukodystrophy (HLD) or other neurological condition.

In some embodiments, provided herein are methods comprising: a) testing a sample from a human subject and determining: i) if said subject is A/A, A/C, or C/C at position 4444 in the Glutamyl-prolyl-tRNA synthetase 1 (EPRS1) gene or mRNA, and/or ii) if said subject expresses only the 1482T version, only the 1482P version, or both the 1482T and 1482P versions, of the EPRS1 protein; and/or iii) if said subject is m6A methylated or m6A unmethylated at A4355 and/or A4464 in said EPRS1 mRNA; and b) generating, transmitting, and/or graphically displaying a report that: i) said subject: A) is A/A at position 4444 in the EPRS1 gene or mRNA; and/or B) expresses only the 1482T version of EPRS1 protein; and/or C) is m6A unmethylated at A4355 and A4464 in said EPRS1 mRNA, and therefore said subject has a neurological disease and/or should be treated with a neurological therapeutic, nutritional therapy, hormone therapy, and/or physical, occupational, and/or speech therapy; and/or ii) said subject: A) is A/C or C/C at position 4444 in the EPRS1 gene or mRNA; and/or B) expresses the 1482P version of EPRS1 protein; and/or C) is m6A methylated at A4355 and A4464 in said EPRS1 mRNA, and that said subject does not have hypomyelinating leukodystrophy (HLD).

In certain embodiments, provided herein are methods comprising: a) receiving and/or reviewing a report that a subject: i) is A/A at position 4444 in the EPRS1 gene or mRNA; and/or ii) expresses only the 1482T version of EPRS1 protein; and/or iii) is m6A unmethylated at A4355 and A4464 in said EPRS1 mRNA; b) treating said subject with a neurological therapeutic, nutritional therapy, hormone therapy, and/or physical, occupational, and/or speech therapy.

In additional embodiments, provided herein are methods of treating a subject with hypomyelinating leukodystrophy (HLD), or other neurological disease, comprising: administering any of the following to said subject: a) an oligonucleotide sequence that at least partially hybridizes to EPRS1 mRNA changing the conformation of said EPRS1 mRNA thereby allowing m6A methylation at positions A4355 and A4464 of said EPRS1 mRNA to occur, optionally wherein said oligonucleotide at least partially hybridizes between positions 4436 and 4464, or between positions 4327 and 4364; and/or b) a Cas targeting system comprising: i) single guide RNA (sgRNA) comprising a targeting sequence (crRNA sequence) and a Cas nuclease-recruiting sequence (tracrRNA), and ii) a fusion protein, or an expression vector encoding said fusion protein, wherein said fusion protein comprises: i) a catalytically-dead Cas enzyme, ii) a nuclear localization signal, and iii) at least a catalytically active portion of a methyltransferase that m6A methylates adenine, wherein said Cas targeting system targets position A4355 and/or A4464 in said EPRS1 mRNA for methylation.

In further embodiments, the neurological therapeutic comprises an HLD therapeutic, and/or wherein said nutritional therapy comprises HLD nutritional therapy, and/or wherein said hormone therapy comprises HLD hormone therapy, and/or wherein said physical, occupational, and/or speech therapy comprises HLD specific physical, occupation, and/or speech therapy, and/or wherein said indicates said subject has HLD. In other embodiments, the neurological therapeutic is selected from: a) a medication for seizures, muscle tightness and/or movement problems; b) an oligonucleotide sequence that at least partially hybridizes to said EPRS1 mRNA changing the conformation of said EPRS1 mRNA thereby allowing m6A methylation at positions A4355 and A4464 of said EPRS1 mRNA to occur, optionally wherein said oligonucleotide at least partially hybridizes between positions 4436 and 4464, or between positions 4327 and 4364; c) a Cas targeting system comprising: i) single guide RNA (sgRNA) comprising a targeting sequence (crRNA sequence) and a Cas nuclease-recruiting sequence (tracrRNA), and ii) a fusion protein, or an expression vector encoding said fusion protein, wherein said fusion protein comprises: i) a catalytically-dead Cas enzyme, ii) a nuclear localization signal, and iii) at least a catalytically active portion of a methyltransferase that m6A methylates adenine, wherein said Cas targeting system targets position A4355 and/or A4464 in said EPRS1 mRNA for methylation.

In some embodiments, the catalytically-dead Cas enzyme comprises a catalytically-dead Cas13b (dCas13b). In other embodiments, the oligonucleotide sequence comprises a Phosphorodiamidate morpholino oligomer (PMO). In further embodiments, the PMO is selected from the following: PMO2; PMO3; PMO4; PMO5; and PMO6. In additional embodiments, the subject is determined to be A/A at position 4444 in the EPRS1 gene or mRNA. In some embodiments, the subject is determined to only express the 1482T version of EPRS1 protein. In additional embodiments, the subject is determined to be m6A unmethylated at A4355 and A4464 in said EPRS1 mRNA. In certain embodiments, the sample comprises a sample type selected from: saliva, serum, plasma, tissue biopsy, and whole blood.

In additional embodiments, provided herein are compositions, kits, and systems comprising: a) an oligonucleotide sequence that at least partially hybridizes to EPRS1 mRNA changing the conformation of said EPRS1 mRNA thereby allowing m6A methylation at positions A4355 and A4464 of said EPRS1 mRNA to occur, wherein said oligonucleotide comprises modified bases that reduce immunogenicity and/or wherein said oligonucleotide is a phosphorodiamidate morpholino oligomer (PMO); optionally wherein said oligonucleotide at least partially hybridizes between positions 4436 and 4464, or between positions 4327 and 4364; and/or b) a Cas targeting system comprising: i) single guide RNA (sgRNA) comprising a targeting sequence (crRNA sequence) and a Cas nuclease-recruiting sequence (tracrRNA), and ii) a fusion protein, or a expression vector encoding said fusion protein, wherein said fusion protein comprises: i) a catalytically-dead Cas enzyme, ii) a nuclear localization signal, and iii) at least a catalytically active portion of a methyltransferase that m6A methylates adenine; wherein said Cas targeting system targets position A4355 and/or A4464 in said EPRS1 mRNA for methylation.

In particular embodiments, the catalytically-dead Cas enzyme comprises a catalytically-dead Cas13b (dCas13b). In other embodiments, the oligonucleotide sequence comprises a Phosphorodiamidate morpholino oligomer (PMO). In other embodiments, the PMO is selected from the following: PMO2; PMO3; PMO4; PMO5; and PMO6. In certain embodiments, the modified base is selected from: 5-methylcytidine (m5C), pseudouridine, and N1-methylpseudouridine.

In some embodiments, provided herein are compositions comprising: a nucleic acid reporter construct, wherein the nucleic acid reporter construct comprises: a) a reporter sequence that generates a detectable signal when expressed in a cell, wherein the reporter sequence is free, or substantially free, of DRACH m6A methylation sites, and b) a nucleic acid sequence of interest with a SNP site, wherein the nucleic acid sequence of interest is operably linked to the reporter sequence and comprises at least one DRACH site, and wherein the at least one DRACH site: i) does not contain the SNP site, and ii) changes its m6A methylation status, but not its sequence, based on a change in the identity of a nucleotide at the SNP site.

In further embodiments, the reporter construct further comprises an expression vector (e.g., plasmid, AAV, adeno-virus), and wherein the reporter sequence and nucleic acid sequence of interest are present in the expression vector. In other embodiments, the the reporter sequence comprises at least a portion of a reporter gene modified to eliminate all, or substantially all, of DRACH sites present. In additional embodiments, the reporter gene is selected from: beta-galactosidase, luciferase, beta-lactamase, alkaline phosphatase, and green fluorescence protein. In particular embodiments, the nucleic acid sequence of interest comprises at least a portion of a target gene selected from those recited in Table 3. In additional embodiments, the SNP of the target gene is selected from those recited in Table 3.

In certain embodiments, the reporter sequence contains zero or one DRACH sites. In other embodiments, the nucleic acid sequence of interest comprises at least two DRACH sites, or at least three DRACH sites or four or more DRACH sites. In other embodiments, the identity of the nucleotide at the SNP site is the minor allele. In particular embodiments, the identity of the nucleotide at the SNP site is known, or suspected of, being linked to a disease in a subject, wherein the subject is optionally a human. In further embodiments, the nucleic acid sequence of interest comprises at least a portion of a target gene, wherein the at least a portion of a target gene comprise a terminal exon of the gene. In some embodiments, the nucleic acid sequence of interest comprises at least a portion of a target gene, wherein the at least a portion of a target gene comprise the two terminal exons of the gene, and optionally an intron from the gene.

In certain embodiments, the nucleic acid construct further comprises a 5′ UTR free of DRACH sites. In other embodiments, the nucleic acid construct further comprises a 3′ UTR free of DRACH sites. In some embodiments, the 3′ UTR free of DRACH sites comprises an SV40 3′ UTR sequence free, or free of all but one, of DRACH sites. In additional embodiments, the change its m6A methylation status is from methylated to non-methylated. In other embodiments, the change its m6A methylation status is from non-methylated to methylated.

In some embodiments, provided herein are methods comprising: a) transfecting a cell with the nucleic acid constructs from above or herein, where the SNP site has a minor allele or disease causing allele, and detecting a first signal from the reporter sequence, and b) transfecting a cell with the nucleic acid constructs above or herein, and where the SNP site has a major allele or wild-type non-disease causing allele, and detecting a second signal from the reporter sequence, and c) comparing the first and second signals, optionally wherein the comparing comprises graphically comparing.

In further embodiment, the methods further comprise: d) identifying the at least one DRACH site in the nucleic acid sequence of interest as having different m6A methylation when the minor allele or disease causing allele is present at the SNP site versus when the major allele or wild-type non-disease causing allele is present at the SNP site. In particular embodiments, the different m6A methylation identified indicates the SNP site changes methylation at the one or more DRACH sites in the nucleic acid sequence of interest.

In some embodiments, provided herein are kits and systems comprising: a) a first nucleic acid construct as describe above or herein where the SNP site has a minor allele or disease causing allele, and b) a second nucleic acid construct above or herein, where the SNP site has a major allele or wild-type non-disease causing allele.

### DESCRIPTION OF THE FIGURES

The patent or application file contains at least one drawing executed in color. Copies of this patent or patent application publication with color drawings will be provided by the Office upon request and payment of the necessary fee.

FIG. 1. Neurologic and genetic characterization of siblings with HLD. (A) Pedigree of the family; arrow indicates proband. (B) Top row (Sibling 1 at 2 yr, 4 mo): Sagittal T1 image (left) demonstrates a thin corpus callosum (CC), which is myelin mature (arrow). Axial T1 image (2^{nd} panel) reveals myelin signal in the anterior limb (short arrow) (ALIC) and in posterior limb (PLIC) (arrow) of internal capsule. Faint myelin signal is present in the frontal lobes (*) and parietal lobes. Axial FLAIR (3^{rd} panel) and T2 (right) images demonstrate abnormally increased signal (arrows) in the PLIC (arrows), frontal lobe white matter (*) and parietal white matter. Second row (Sibling 1 at 9 yr, 3 mo): Sagittal T1 image (left) reveals thin CC with loss of myelin signal (arrow). Frontal lobe white matter (*) has lost signal on T1 axial (2^{nd} panel) and is unchanged on FLAIR (3^{rd} panel) and T2 (right) axial images. ALIC (short arrows) has thinned on T1, FLAIR, and T2 weighted axial images, while PLIC has thinned on T1 and remains abnormal in signal on T2 and FLAIR. Both the ALIC and genu of internal capsule (short arrows) have lost myelin signal on T1, FLAIR, and T2 weighted axial images. Third row (Sibling 2 at 3 yr, 2 mo): Sagittal T1 image (left) demonstrates a thin CC with trace myelin in splenium (arrow). Axial T1 (2^{nd} panel) also reveals trace myelin signal (arrows) in the anterior limb of the internal capsule (ALIC) and in the corticospinal tract (CSP) in the mid-third of the posterior limb of internal capsule (PLIC). Absence of myelin signal is seen in the frontal lobes (*) and parietal lobes on T1 (2^{nd} panel) axial images. Axial FLAIR (3^{rd} panel) and T2 (right) images demonstrate abnormally increased signal (arrows) in the PLIC (arrows), frontal lobe white matter (*) and parietal white matter. Bottom row (Sibling 2 at 8 yr): Sagittal T1 (left) reveals persistently thin CC with loss of splenium myelin (arrow). Frontal lobe white matter (*) is lower in signal on T1 axial (2^{nd} panel) and unchanged on FLAIR (3^{rd} panel) and T2 (right) axial images. PLIC abnormal signal is unchanged. ALIC (arrows) has lost myelin signal on T1, FLAIR, and T2 weighted axial images. (C) Sanger sequencing of DNA isolated from immortalized LCLs generated from unaffected control 3 (left), carrier parent (center), and affected sibling (right). EPRS1 c4444C>A variant position (*).

FIG. 2. Dual post-transcriptional mechanisms dictate reduced expression of Pro1482Thr EPRS1 in siblings with HLD. (A) Domain structure of EPRS1 including ProRS sub-domains and Pro1482Thr substitution site (top). Crystal structure of ProRS dimer of human EPRS1 highlighting Pro1482Thr substitution site (bottom). (B) Cell tRNA charging activities of ProRS (top) and FARS1 (bottom) were determined in LCL lysates by charging yeast tRNA with [¹⁴C]Pro and [¹⁴C]Phe, respectively. (C) Recombinant WT and Pro1482Thr (P1482T) mutant human 3×FLAG-EPRS1 were expressed in HEK293F cells, and ProRS- and GluRS-specific tRNA charging activities determined by incorporation of [¹⁴C]Pro and [¹⁴C]Glu, respectively, into yeast tRNA. (D) EPRS1 in LCL lysates was determined by immunoblot and densitometry. Mean±SD, n=6 biological replicates. (E) EPRSJ mRNA was determined by RT-qPCR and normalized to GAPDH mRNA. Mean±SD, n=3 biological replicates. (F) Polysome profiling comparing LCLs from female sibling and mother (left), and comparing LCLs from male sibling and father (right). (G) Metabolic labeling of LCLs with [³⁵S]Cys/Met, followed by SDS-PAGE and autoradiography. (H) Dimerization status of ProRS domain. Recombinant WT (left) or Pro1482Thr ProRS purified from E. coli and analyzed by size-exclusion chromatography. (I) Incorporation of mutant EPRS1 in the MSC was determined following transfection of pCMV vectors expressing FLAG-tagged, full-length WT and Pro1482Thr mutant EPRS1 into HEK293T cells. Tagged protein was purified with anti-FLAG resin, and bound proteins eluted and subjected to immunoblot. (J) Time course of EPRS1 expression in cycloheximide-treated LCLs (left). Densitometric quantitation of EPRS1 expression (right, representative of n=3 indepen-dent experiments). (K) Determination of EPRS1 mRNA translation in LCLs by poly-some profiling. Mean±SD, n=3 biological replicates for control LCL and n=6 pooled female and male biological replicates for parent and sibling LCLs. (L) Nuclear and cytoplasmic fractions of LCL lysates probed for EPRS1 mRNA and 18S rRNA by RT-qPCR. Mean±SD, n=6 pooled biological replicates for control LCLs, n=4 pooled female and male biological replicates for parent and sibling LCLs; p values are from unpaired two-tailed t test). or *p<0.05, ***p<0.001, ****p<0.0001; ns, not significant.

FIG. 3. Role of m6A modification in EPRS1 mRNA nuclear export and expression. (A) Role of terminal intron in EPRS1 reporter expression. hRLuc reporters containing terminal exons 31 and 32 (hRLuc-EE), or terminal exons with intervening intron, 131 (hRLuc-EIE), or hRLuc-EIE reporter with exon 30 and intervening intron, RBGI (hRLuc-E-RBGI-EIE); all reporters with or without C4444A mutation (left). Following transfection into HEK293T cells, hRLuc activities were normalized to FLuc (right). (B) Effect of replacement of 131 with alternate intron, cI (hRLuc-EcIE, left). Normalized reporter activities of reporters with and without C4444A mutation (right). (C) Experimentally validated m⁶A sites in exons 31-32 in human EPRS1 pre-mRNA. (D) Following siRNA-mediated knockdown of METTL3 in HEK293T cells EPRS1 expression in lysates was determined by immunoblot. (E) YTHDC1 knockdown in LCLs decreased EPRS1 expression (left) and EPRS1 mRNA nuclear retention (center), but not EPRS1 mRNA amount (right). (F) Effect of inhibition of NXF1 in TREX/TREX-2 (left), GANP in TREX-2 (2^{nd} from left), CRM1 in eIF4E-CRM1 (3^{rd} from left) and IPMK in Sp.AlyREF (right) export pathways on EPRS1 expression in HEK293T cells. (G) Influence of YTHDF readers on EPRS1 expression. LCLs were subjected to siRNA-mediated knockdown targeting YTHDF1 (left), YTHDF2 (center), and YTHDF3 (right).

FIG. 4. Effect of C4444A mutation on m⁶A modification of EPRS1 reporter expression and processing of endogenous EPRS1 mRNA in patient LCLs. (A) Elimination of DRACH sequences in hRLuc reporter (DRACH⁻, left) enhances inhibition of expression by C4444A mutation (right). (B) Normalized expression of DRACH-hRLuc reporter in HEK293T cells following pairwise mutation of m⁶A sites. (C) Detection of m⁶A modification of reporters by anti-m⁶A RNA immunoprecipitation (RIP). (D) Detection of m⁶A modification WT and C4444A variant EPRS1 mRNA in patient and control LCLs by anti-m⁶A RIP-qPCR. (E-G) Detection of EPRS1 mRNA binding to YTHDC1 (E), YTHDC2 and YTHDF2 (F), an YTHDF1 and YTHDF3 (G) by RIP-qPCR in patient and control LCLs.

FIG. 5. Identification of m⁶A sites defective in hRLuc reporter bearing c.4444C>A variant in EPRS1 c.4444C>A mRNA. (A) Folding model of exons 31 and 32 of WT (top) and C4444A variant (bottom) EPRS1 mRNA. Stems near C4444 site (red), near 16727 m⁶A site (green), and 16728 m⁶A site (blue) are highlighted. (B) Effect of mutations in the 5-bp stem surrounding C4444A site on hRLuc reporter expression in 293T cells. Mean±SD, n=14; p values from unpaired two-tailed t test; ns, not significant. (C) Effect of mutations in the stems surrounding 16727 and 16728 m⁶A sites on hRLuc reporter expression in 293T cells. Mean±SD, n=14; p values from unpaired two-tailed t test; ns, not significant.

FIG. 6. Targeted rescue of expression of c.4444C>A EPRS1 variant in patient LCLs. (A) Folding model of C4444A variant EPRS1 mRNA exons 31-32 with antisense PMOs highlighted. (B) LCLs from controls, parents, and female (top) and male (bottom) siblings were incubated with control or selected pairs of antisense PMOs, and EPRS1 expression determined by immunoblot. (C) Effect of PMOs on m⁶A modification of EPRS1 mRNA in WT and patient LCLs was determined by m⁶A-RIP-qPCR. Mean±SD, n=4 pooled male and female siblings; p values from unpaired two-tailed t test. (D) Application of targeted RNA methylation (TRM) system. dCas13b with an N-terminus NLS is ligated to METTL3 or inactive METTL3^{mut} (left). crRNAs with direct repeats were targeted 8 or 14 nt upstream of the three m6A sites in EPRS1 mRNA exons 31-32 (right). (E) LCLs from controls and female (top) and male (bottom) siblings were co-transfected every 2 d with crRNAs and chimeric dCas13b linked to METTL3 or METTL3^{mut} for 6 d, and EPRS1 expression determined by immunoblot. (F) Effect of TRM on m⁶A modification of EPRS1 mRNA in WT and patient LCLs was determined by m⁶A-RIP-qPCR. Mean±SD, n=4 pooled male and female siblings; p values from unpaired two-tailed t test.

FIG. 7. Schematic of defective processing of EPRS1 mRNA bearing c.4444C>A variant. Processing of wild-type (top) and C4444A HLD variant EPRS1 mRNA (bottom).

FIG. 8. Evolutionary conservation of EPRS1 Pro1482, and 5′RACE analysis of influence of the c.4444C>A mutation on splicing and polyadenylation, related to FIG. 2. (A) Clustal Omega generated alignment of reference sequences of the EPRS1 protein in Drosophila and higher organisms or ProRS in yeast and lower organisms. Arrows indicates the position of Pro1482 and Zn2+ binding sites in human EPRS1. Alignment by Clustal Omega was visualized using Unipro Ugene. (B) Schematic representation of 3′ RACE. Exon 31-32 boundary is shown. Point mutation is indicated by red line. The 52-nt Q0-QI-TTTTTT primer is used to reverse transcribe cellular mRNAs and gene-specific primer 1 (GSP1) and primer 2 (GSP2) are used in sequential amplifications to generate sequence-specific product. (C) Chromatogram of exon 31-32 of EPRS1 mRNA showing boundary and polyA tail.

FIG. 9. (A) Effect of replacement of 131 with alternate intron, cI, in triple-exon reporter (hRLuc-E-RBGI-EcIE, left). Normalized reporter activities of reporters with and without C4444A mutation (right). (B) Non-synergy of a 5′intron (e.g., cI) in the 5′UTR of hRLuc-EE reporter (left, top two schematics) with the HLD-causing c.4444C>A variant (right). Evidence of synergy only when an intervening intron (e.g., 131) is present between exons 31 and 32. (left, bottom two schematics), highlights a specific role of this exon-exon junction.

FIG. 10. Mutations introduced into hRLuc to inactivate DRACH sites, related to FIG. 3. Synonymous mutations (boxed) were introduced except for a requisite Thr-to-Ser mutation (boxed with labels). The top sequence in FIG. 10 is a Renilla luciferase gene (lRLuc) with a number of DRACH sites. The bottom sequence (DLRLT1845) is the same sequence at the top sequence, except 7 of the 8 DRACH sites are changed to be non-DRACH sites.

FIG. 11. Identification of functional m6A sites in Exons 31-32 of EPRS1 mRNA in the U87-MG glioblastoma cell line, related to FIG. 4. (A) Normalized expression of DRACH-hRLuc reporter in U87-MG cells following pairwise mutation of m6A sites. (B) Detection of m6A modification of reporters by anti-m6A RNA immunoprecipitation (RIP).

FIG. 12. Role of 5-bp stem in expression of c.4444C>A EPRS1 variant in patient LCLs, related to FIG. 5. Effect of mutations in the 5-bp stem surrounding C4444A site on hRLuc reporter expression in 293T cells. Mean±SD, n=8; p values from unpaired, two-tailed t test; ns, not significant.

FIG. 13. Effect of PMOs on variant EPRS1 expression and EPRS1 mRNA amount, related to FIG. 6. (A) LCLs from controls, parents, and female (left) and male (right) siblings were incubated with control or singleton (B) Effect of pairwise addition of PMOs on EPRS1 mRNA in LCLs from controls and HLD siblings as determined by RT-qPCR. Mean±SD, n=4 male and female siblings pooled. (C) Effect of METTL3-dCas13b on EPRS1 mRNA on EPRS1 mRNA in LCLs from controls and HLD siblings as determined by RT-qPCR. Mean±SD, n=4 male and female siblings pooled.

FIG. 14 shows the nucleic acid sequences of the following: A) gRNA HLD16727-8 (SEQ ID NO:1), B) gRNA HLD16727-14 (SEQ ID NO:2), C) gRNA WT16727-8 (SEQ ID NO:3), D) gRNA WT16727-14 (SEQ ID NO:4), E) gRNA 16728-8 (SEQ ID NO:5), F) gRNA 16728-14 (SEQ ID NO:6), G. gRNA 16726-8 (SEQ ID NO:7), H) gRNA 16726-14 (SEQ ID NO:8), I) PMO 5-29 (PMO2) (SEQ ID NO:9), J) PMO 7-31 (PMO3) (SEQ ID NO:10), K) PMO 217-241 (PMO5) (SEQ ID NO:11), L) PMO 215-239 (PMO4) (SEQ ID NO:12), and M) PMO 8-26/35-40 (PMO6) (SEQ ID NO:13). All of these gRNAs showed effects to varying degrees in a reporter assay (not shown), but we further pursued the names highlighted in red. Brown: spacer sequence unique to a guide; blue: direct repeat common to all. The regions that the various PMO's span are as follows: PMO2: spans 4328-4352; PMO3: spans 4330-4354; PMO4: spans 4437-4461; PMO5: spans 4439-4463; PMO6: spans 4331-4349 and 4358-4363, predicted to bulge 4350-4357 that contains site 16728 (m6A site 4355).

FIG. 15 shows the amino acid sequence of METTL3-dCas13b Fusion Construct (SEQ ID NO:17). The dCas13b-METTL3 construct (sequence in this figure) and -METTL3.mut constructs (has an “A” and the underlined “D” in FIG. 15) are Addgene products (Plasmid #155366 and Plasmid #157854).

FIG. 16. Predicted changes in base-pairing of m⁶A-sites driven by health status-associated single nucleotide variants (SNV). RNAfold analysis of a selected 250-nt RNA sequence (see Methods for details) in VHL (A) and TSC2 (B) transcripts, without (left) or with (right) a ClinVar-curated SNV. Highlighted are DRACH site (green), m⁶Ad-SNV (orange), and stop codon (light blue) nucleotides. DRACH sites are enumerated 5′ to 3′ and labeled in green if 3 or more nucleotides are unpaired or in magenta if 3 or more nucleotides are paired. DRACH sites predicted to be altered by m⁶Ad-SNV are boxed on both reference and alternate sequences.

FIG. 17A shows a construct composed of: i) a DRACH free 5′ UTR, ii) DRACH-reduced luciferase gene (only 1 DRACH site preserved out of 8), iii) a circled site that is a place for a target sequence of interest with one or more DRACH sites and having a SNP site, and iv) an SV40 3′UTR with DRACH sites mutated to remove them). FIG. 17B shows the circled site in FIG. 17A, showing the location of DRACH sites and SNP site in the target sequence.

The top sequence in FIG. 18 (3_UTR_SV40) is a natural SV40 3′ UTR sequence with five DRACH sites. The bottom sequence (3_UTR_DL) is the same sequence at the top sequence, except the five DRACH sites are changed to be non-DRACH sites.

### DETAILED DESCRIPTION

The present invention relates to methods, kits, and compositions for testing a sample from subject (e.g., human) and determining: i) if said subject is A/A, A/C, or C/C at position 4444 in the Glutamyl-prolyl-tRNA synthetase 1 (EPRS1) gene or mRNA, and/or ii) if said subject expresses only the 1482T version, only the 1482P version, or both the 1482T and 1482P versions, of the EPRS1 protein; and/or iii) if said subject is m6A methylated or m6A unmethylated at A4355 and/or A4464 in said EPRS1 mRNA; and determining that the subject has hypomyelinating leukodystrophy (HLD) or other neurological condition.

In work conducted during the development of embodiments herein, we have identified a novel homozygous c.4444C>A; p.Pro1482Thr missense variant in EPRS1 in two siblings presenting with microcephaly, severe global developmental delays, progressive gait disturbance, spasticity, and hypomyelination on brain MRI consistent with childhood-onset HLD15 (OMIM 617951). We show the variant does not alter specific charging activity of EPRS1, but markedly diminishes its expression by dual post-transcriptional mechanisms involving reduced m⁶A modification of variant EPRS1 mRNA.

The present disclosure is not limited as to how m6A methylation is detected. Such m6A methylation detection is known in the art. For example, one could use the service from ArrayStar (Rockville Maryland) or the kit from EpigenTek (Farmingdale, NY).

The present disclosure is not limited with regard to how position 4444 (C>A) in the EPRS1 gene or mRNA is detected to see if a subject has one or two alleles of C. In some embodiments, detection involves measurement or detection of a characteristic of a non-amplified nucleic acid, amplified nucleic acid, a component comprising amplified nucleic acid, or a byproduct of the amplification process, such as a physical, chemical, luminescence, or electrical aspect, which correlates with amplification (e.g. fluorescence, pH change, heat change, etc.). In some embodiments, fluorescence detection methods are provided for detection of amplified or non-amplified EPRS1 nucleic acid. In certain embodiments, various detection reagents, such as fluorescent and non-fluorescent dyes and probes are employed. For example, the protocols may employ reagents suitable for use in a TaqMan reaction, such as a TaqMan probe; reagents suitable for use in a SYBR Green fluorescence detection; reagents suitable for use in a molecular beacon reaction, such as molecular beacon probes; reagents suitable for use in a scorpion reaction, such as a scorpion probe; reagents suitable for use in a fluorescent DNA-binding dye-type reaction, such as a fluorescent probe; and/or reagents for use in a LightUp protocol, such as a LightUp probe. In some embodiments, provided herein are methods and compositions for detecting and/or quantifying a detectable signal (e.g. fluorescence) from EPRS1 target nucleic acid. Thus, for example, methods may employ labeling (e.g. during amplification, post-amplification) amplified nucleic acids with a detectable label, exposing partitions to a light source at a wavelength selected to cause the detectable label to fluoresce, and detecting and/or measuring the resulting fluorescence. Fluorescence emitted from label can be tracked during amplification reaction to permit monitoring of the reaction (e.g., using a SYBR Green-type compound), or fluorescence can be measure post-amplification. In some embodiments, the 4444 position in EPRS1 is detected with methods described in U.S. Pat. No. 9,856,536, which is herein incorporated by reference in its entirety.

In some embodiments, detection of EPRS1 nucleic acids employs one or more of fluorescent labeling, fluorescent intercalation dyes, FRET-based detection methods (U.S. Pat. No. 5,945,283; PCT Publication WO 97/22719; both of which are incorporated by reference in their entireties), quantitative PCR, real-time fluorogenic methods (U.S. Pat. No. 5,210,015 to Gelfand, U.S. Pat. No. 5,538,848 to Livak, et al., and 5,863,736 to Haaland, as well as Heid, C. A., et al., Genome Research, 6:986-994 (1996); Gibson, U. E. M, et al., Genome Research 6:995-1001 (1996); Holland, P. M., et al., Proc. Natl. Acad. Sci. USA 88:7276-7280, (1991); and Livak, K. J., et al., PCR Methods and Applications 357-362 (1995), each of which is incorporated by reference in its entirety), molecular beacons (Piatek, A. S., et al., Nat. Biotechnol. 16:359-63 (1998); Tyagi, S. and Kramer, F. R., Nature Biotechnology 14:303-308 (1996); and Tyagi, S. et al., Nat. Biotechnol. 16:49-53 (1998); herein incorporated by reference in their entireties), Invader assays (Third Wave Technologies, (Madison, Wis.)) (Neri, B. P., et al., Advances in Nucleic Acid and Protein Analysis 3826:117-125, 2000; herein incorporated by reference in its entirety), nucleic acid sequence-based amplification (NASBA; (See, e.g., Compton, J. Nucleic Acid Sequence-based Amplification, Nature 350: 91-91, 1991.; herein incorporated by reference in its entirety), Scorpion probes (Thelwell, et al. Nucleic Acids Research, 28:3752-3761, 2000; herein incorporated by reference in its entirety), partially double-stranded linear probes (Luk, K.-C., et al, J. Virological Methods 144:1-11, 2007; herein incorporated by reference in its entirety), capacitive DNA detection (See, e.g., Sohn, et al. (2000) Proc. Natl. Acad. Sci. U.S.A. 97:10687-10690; herein incorporated by reference in its entirety), etc.

Target EPRS1 nucleic acid molecules (e.g., amplified EPRS1 mRNA) may be analyzed by any number of techniques to determine the presence of, amount of, or identity of the molecule. Non-limiting examples include sequencing, mass determination, and base composition determination. The analysis may identify the sequence of all or a part of the amplified nucleic acid (e.g., containing the 4444 C/A mutation) or one or more of its properties or characteristics to reveal the desired information.

Illustrative non-limiting examples of nucleic acid sequencing techniques include, but are not limited to, chain terminator (Sanger) sequencing and dye terminator sequencing, as well as “next generation” sequencing techniques. A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the systems, devices, and methods employ parallel sequencing of partitioned amplicons (PCT Publication No: WO2006084132 to Kevin McKernan et al., herein incorporated by reference in its entirety). In some embodiments, DNA sequencing is achieved by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties) the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), Illumina Single base sequencing technology, the Lynx massively parallel signature sequencing technology (Brenner et al. (2000). Nat. Biotechnol. 18:630-634; U.S. Pat. Nos. 5,695,934; 5,714,330; herein incorporated by reference in their entireties) and the Adessi PCR colony technology (Adessi et al. (2000). Nucleic Acid Res. 28, E87; WO 00018957; herein incorporated by reference in its entirety).

The present disclosure is not limited with the methods used to determine if the human EPRS1 protein has a threonine at position 1482 (1482T) or proline at position 1482 (1482P), and not limited by the methods used to determine the amount of 1482T or 1482P that is expressed. For example, these EPRS1 variants can be measured using any suitable methodology, including but not limited, to mass spectrometry, HPLC/UV, HPLC/Vis, LC/MS/MS, immunological detection methods. In other embodiments, the variants of EPRS1 are measured using: 1) a sandwich immunoassay (e.g., monoclonal, polyclonal and/or DVD-Ig sandwich immunoassays or any variation thereof (e.g., monoclonal/DVD-Ig or DVD-Ig/polyclonal), including chemiluminescence detection, radioisotope detection (e.g., radioimmunoassay (RIA)) and enzyme detection (e.g., enzyme immunoassay (EIA) or enzyme-linked immunosorbent assay (ELISA) (e.g., Quantikine ELISA assays, R&D Systems, Minneapolis, Minn.))), 2) a competitive inhibition immunoassay (e.g., forward and reverse), 3) a fluorescence polarization immunoassay (FPIA), 4) an enzyme multiplied immunoassay technique (EMIT), 5) a bioluminescence resonance energy transfer (BRET), 6) a homogeneous chemiluminescent assay, 7) a SELDI-based immunoassay, 8) chemiluminescent microparticle immunoassay (CMIA) and 9) a clinical chemistry colorimetric assay (e.g., IMA, creatinine for eGFR determination and LC-MS/MS). (See, e.g., Tietz Textbook of Clinical Chemistry and Molecular Diagnostics. 4th Edition, edited by C A Burtis, E R Ashwood and D E Bruns, Elsevier Saunders, St. Louis, Mo., 2006.). In certain embodiments, the methods described in U.S. Pat. No. 9,856,535 (herein incorporated by reference) are employed.

Further, if an immunoassay is being utilized, any suitable detectable label as is known in the art can be used. For example, the detectable label can be a radioactive label (such as 3H, 125I, 35S, 14C, 32P, and 33P), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous or heterogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

### EXAMPLES

### Example 1

Multiple pathogenic gene variants cause hypomyelinating leukodystrophy (HLD), characterized by defective central nervous system myelination, and disturbed motor and cognitive function. Exome sequencing of two siblings with severe cognitive and motor impairment and progressive hypomyelination on brain magnetic resonance imaging, characteristic of HLD, revealed homozygosity for a missense variant in EPRS1 (c.4444C>A; p.Pro1482Thr), which encodes glutamyl-prolyl-tRNA synthetase. Patient lymphoblastoid cell lines exhibit reduced EPRS1 protein due to dual defects in nuclear export and cytoplasmic translation of variant EPRS1 mRNA. Variant mRNA reveals reduced METTL3 methyltransferase-mediated “writing” of m⁶A at two variant-distal sites, and reduced “reading” by YTHDC1 and YTHDF1/3, required for efficient mRNA nuclear export and translation, respectively. The cryptic m⁶A sites are exposed by antisense morpholinos or by site-directed introduction of m⁶A by METTL3-dCas13b, thereby restoring EPRS1 expression in patient cells. The work reveals an etiologic mechanism by which a pathological m⁶A-distal single-nucleotide variant depresses post-transcriptional gene expression. It is noted that mRNA positions here are numbered according to Genomic m6A site # from m6A-Atlas version 1.0

### Methods

### Patient Recruitment

Both patients are followed in a specialized Neurogenetics Clinic at The Hospital for Sick Children (Toronto, Canada). Extensive genetic and non-genetic investigations were initially non-diagnostic (Table 2).

TABLE 2
Ancillary clinical determinations of siblings with
homozygous c.4444C > A; p.(Pro1482Thr) EPRS1 variant

| | Proband (Sibling 1) | Sibling 2 |
|---|---|---|
| Metabolic laboratory | Ammonia, lactate, vitamin B12, vitamin | Normal |
| investigations | E, homovanillic acid (HVA)/vanillylman- | |
| | delic acid (VMA), plasma amino acids, | |
| | very long chain fatty acids, phytanic acid, | |
| | urine organic acids and mitochondrial | |
| | respiratory chain complex enzyme | |
| | activity ratios (in fibroblasts) all normal | |
| SNP-based chromosomal | Maternally inherited 248 kb loss at 6q16.1 | A maternally inherited 248 kb loss at |
| microarray analysis | of no apparent clinical significance, as | 6q16.1, a 22 kb loss at 9q33.1, and a |
| | well as multiple genomic regions with | paternally inherited 388 kb gain at |
| | absence of heterozygosity. | 10q26.13, which were of no apparent |
| | | clinical significance. There were also |
| | | multiple genomic regions with |
| | | absence of heterozygosity. |
| Genetic testing | Targeted genetic testing for Pelizaeus- | Targeted genetic testing for Pelizaeus- |
| | Merzbacher disease and related HLDs | Merzbacher disease and related HLDs |
| | (sequencing and deletion/duplication | (sequencing and deletion/duplication |
| | analyses of PLP1, GJC2, AIMP1, and | analyses of PLP1, AIMP1, HSPD1, and |
| | HSPD1) and sequencing and deletion | FAM126A) was negative. Sequencing |
| | testing of the mitochondrial genome was | and deletion testing of the |
| | negative | mitochondrial genome was negative |
| Clinical exome | Homozygous for a novel predicted | Homozygous for a novel predicted |
| sequencing | damaging missense variant c.4444C > A; | damaging missense variant c.4444C > A; |
| | p.(Pro1482Thr) in the gene EPRS1 | p.(Pro1482Thr) in the gene EPRS1 |
| | [NM_004446.2:]. Both parents and | [NM_004446.2:]. Both parents and |
| | unaffected brother are heterozygous. | unaffected brother are heterozygous. |

This study was approved by the Research Ethics Board of The Hospital for Sick Children. The parents provided written informed consent to participate in this study, including publication of clinical details and brain imaging.

### Exome Sequencing

Quad-based whole-exome sequencing performed in a Clinical Laboratory Improvement Amendments (CLIA)-approved laboratory (GeneDx) revealed that the affected siblings are homozygous for the missense variant c.4444C>A (p.P1482T) in EPRS1 (GenBank: NM_004446.2). This variant was not observed in the >130,000 unrelated individuals in the Genome Aggregation Database (gnomad. followed by broadinstitute.org/). Mean depth of sequence coverage was 98×, with 97.6% of the defined target region with read depth at least 10×, including 100% of the coding region of EPRS1.

### Generation of Lymphoblastoid Cell Lines

Immortalized lymphoblastoid cell lines were generated from both affected patients, both carrier parents, and an unaffected control subject (Control 3) at The Centre for Applied Genomics, The Hospital for Sick Children (Toronto, Canada). Lymphoblast cultures were grown in RPMI 1640 with or without penicillin/streptomycin and 15% fetal bovine calf serum and transformed with Epstein-Barr virus according to standard methods. After immortalization, lymphoblastoid cells were grown in RPMI 1640 supplemented with 15% fetal calf serum and a mix of penicillin/strepto-mycin/amphotericin B and L-glutamine in a 37 °C incubator stabilized at 5% CO₂. Additional race and ethnicity-matched control LCLs (Controls 1, 2) from unaffected subjects were obtained from Dr. Charis Eng (Genomic Medicine Biorepository, Cleveland Clinic).

tRNA Aminoacylation Activity

tRNA aminoacylation activity was measured in a 20 ml reaction volume in HEPES assay buffer (20 mm HEPES, pH 8.0, 100 mm NaCl, 5 mm MgCl₂, 3 mm ATP, and 1 mm DTT) supplemented with 150 mm 1-[¹⁴C] labeled amino acid as show and 1 mg of total yeast tRNA. The reactions were preequilibrated to 37 °C prior to initiation by addition of ProRS lysate to a final concentration of 2.5 mg of total protein. Reactions were incubated at 37 °C for 15 min, and 15 ml aliquots were collected and spotted on glass filters (Whatman GF/C™) presoaked with 5% trichloroacetic acid (TCA). Filters were washed 3×1 ml with 5% TCA followed by 2×1 ml washes with 100% ethanol. Filters were dried in hybridization oven at 60 °C for 10 min. Radioactivity on filters was quantified by liquid scintillation counting (TRI-CARB 1900TR, PerkinElmer Life Sciences).

### 3′ RACE Analysis

3′ RACE was done as described (Scotto-Lavino et al., 2006). Briefly, total RNA was isolated from mouse brain using Trizol, and cDNA was generated by reverse transcription followed by PCR using SuperScript III One-Step RT-PCR System (Invitrogen). The 52-nt (Q₀-Q₁-T) CCAGTGAGCAGAGTGACGAGGACTCGAG-CTCAAGCTTTTTTTTTTTTTTTTT (SEQ ID NO:14) primer was used to reverse transcribe cellular mRNAs. Primers Q₀-CCAGTGAGCAGAGTGACG (SEQ ID NO:15), Q₁-GAGGACTCGAGCTCAAGC (SEQ ID NO:16) and gene-specific primers were used in sequential amplifications to generate sequence-specific product.

### Polysome Profiling

Isolation of ribosome-rich, translationally-active and ribosome-poor, inactive mRNA pools was done by sucrose gradient fractionation. Cycloheximide (100 μg/mL) was added to 10⁶ HEK293F cells for 20 min, and cells collected by low-speed centrifugation and washed twice with cycloheximide-containing, ice-cold PBS. Cell pellets were suspended in 350 μL of lysis buffer (10 mM Tris pH 7.4, 5 mM MgCl₂, 100 mM KCl, 1% Triton X-100, 0.5% deoxycholate, 2 mM DTT, 100 μg/mL cycloheximide, and RNAse inhibitor) and incubated for 5 min on ice. The lysates were centrifuged at 15,000 g for 10 min and supernatants collected. RNase inhibitor (5 uL, 40 U/mL) and cycloheximide (100 μg/mL) were added to 50 ml each of freshly prepared 10% and 50% sucrose gradient solutions (20 mM HEPES pH 7.4, 100 mM KCl, 5 mM MgCl₂, and 2 mM DTT) just before use. Lysates were loaded onto the sucrose gradient and centrifuged at 29,000 rpm for 4 h, and 8 fractions of about 1 mL were collected and combined. Fractions containing light ribonucleoproteins, 40S, 60S, and 80S ribosomes formed the translationally-inactive pool, and heavy polysome fractions formed the translationally-active pool.

### Measurement of Protein Stability

LCLs cells (1×10⁶ cells) were incubated with cycloheximide (50 ug/mL) in 4 mL of DMEM for indicated time points, harvested, and lysed. Lysates were probed by immunoblot for tubulin and EPRS1 antibodies.

### Results

Evaluation of Siblings with Global Developmental Delay and Neurological Impairment

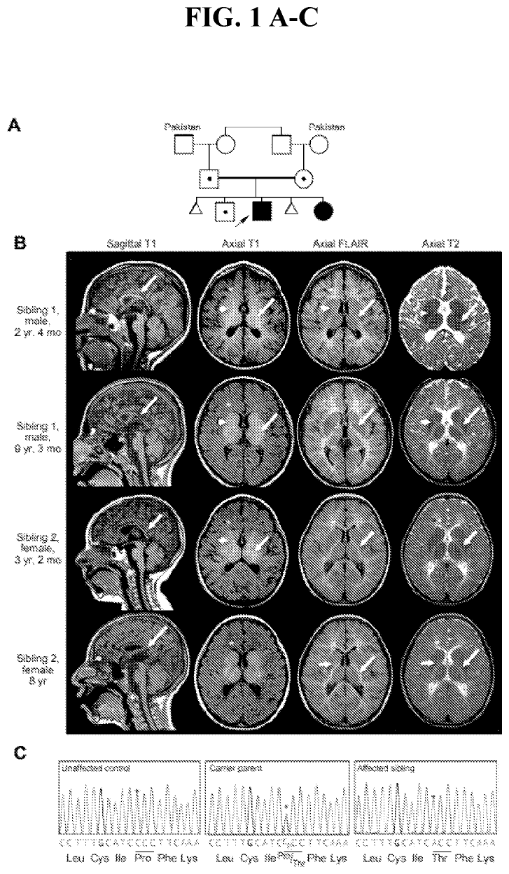

Two siblings born to healthy consanguineous parents of Pakistani descent were evaluated in the Neurogenetics clinic at the Hospital for Sick Children (FIG. 1A). The Proband, Sibling 1, is an 18-year-old male with severe global developmental delays, ataxia, microcephaly, rotatory nystagmus, axial hypotonia, and progressive bilateral lower limb spasticity on serial neurological examinations. At 18 years he was fully dependent on a wheelchair for ambulation and his cognitive function was estimated to be at the level of a two year-old. Serial magnetic resonance imaging (MRI) of the brain at 2 and 9 years showed microcephaly with diffuse supratentorial and infratentorial volume loss, thinning of the corpus callosum, and global hypomyelination with progressive myelin loss (FIG. 1B). The proband's sister, Sibling 2, exhibited similar clinical features. At 16 years, her primary mode of ambulation was a wheelchair, but she could use a walker on level ground. She was less severely affected cognitively, but like her brother was completely dependent for all activities of daily living. Her medical history was otherwise unremarkable until age 15 when she became non-ambulatory and was also diagnosed with premature ovarian insufficiency for which she is treated with levonorgestrel-ethinyl estradiol. MRI of the brain at 3 and 8 years showed microcephaly and global hypomyelination with progressive myelin loss similar to Sibling 1 (FIG. 1B). Further clinical details are available in Table 1.

TABLE 1
Clinical characteristics of siblings with homozygous c.4444C > A; p.(Pro1482Thr) EPRS1 variant

| | Proband (Sibling 1) | Sibling 2 |
|---|---|---|
| Sex (M/F) | Male | Female |
| Age at last review | 18 years | 16 years |
| Pregnancy | Unremarkable | Symmetric intrauterine growth restriction |
| Birth/gestational age | Uncomplicated spontaneous vaginal | Vaginal delivery after induction of labor at |
| | delivery at 39 weeks | 37 weeks |
| Birth weight | 2.9 kg (−2.3 SD)* | N/A |
| Birth length | 46.5 cm (−3.6 SD)* | N/A |
| Birth head | 31.75 cm (−3.1 SD)* | N/A |
| circumference | | |
| Development | Severe global delays - sat at 8 months, | Severe global delays - sat at 8 months, |
| | walked at 34 months, spoke first words at | walked at 21 months, spoke first words at |
| | 18 months. Deteriorated at age 4 years | 18 months. Developed rotatory nystagmus at 2 |
| | during a prolonged diarrheal illness, | years during a prolonged diarrheal illness. |
| | with onset of rotatory nystagmus | She became non-ambulatory at 15 years |
| | and loss of ambulation | |
| Current Motor | Fully dependent on a wheelchair for | Primary mode of ambulation was a wheelchair |
| Function | ambulation, with ongoing slow | but able to use a walker. Progressive |
| | progression of spasticity and weakness | bilateral lower limb spasticity and weakness |
| Current Language | Spoke with single words and had an | Spoke with single words or short sentences |
| Function | estimated vocabulary of up to 25 words | |
| Current Functional | Dependent for all activities of daily living | Dependent for all activities of daily living |
| Status | | |
| Current Cognitive | Estimated to be at the level of <2 years, | Able to name numbers, letters, colors, and |
| ability | could follow simple one step commands | spell her name |
| Seizures | None | Two “episodes” at age 12 but none since |
| Growth | Failure to thrive | Poor weight gain and constipation |
| Endocrine | No reported issues | Achieved menarche at 13 years, diagnosed |
| | | with premature ovarian insufficiency at 15 |
| | | years, treated with levonorgestrel-ethinyl |
| | | estradiol |
| Current head | 18 years, 50 cm (−3.5 SD) | 16 years, 47 cm (−4.8 SD) |
| circumference | | |
| Current height | 18 years, 163.5 cm (5^{th} centile) | 16 years, 141 cm (−3.1 SD) |
| Current weight | 18 years, 36.4 kg (−4 SD) | 16 years, 31.4 kg (−4.2 SD) |
| Cranial nerve exam | Pronounced rotary nystagmus and saccadic | Pronounced rotary nystagmus and saccadic |
| | smooth pursuit, normal extraocular | smooth pursuit, normal extraocular |
| | movements, dysarthria, no bulbar signs | movements, left esotropia, dysarthria, no |
| | | bulbar signs |
| Motor Exam | Axial hypotonia and appendicular hypertonia | Axial hypotonia and appendicular hypertonia |
| | with spasticity. Able to generate movements | with spasticity. Able to generate movements |
| | against gravity but not against resistance. | against gravity but clear weakness with added |
| | Hyperreflexic with extensor plantar | resistance. Hyperreflexic, with extensor |
| | response and clonus bilaterally | plantar response and clonus bilaterally |
| Coordination/ | Head titubation, ataxia and action tremor | Head titubation, ataxia and action tremor |
| Cerebellar exam | of both hands, with pronounced dysmetria | of both hands, with pronounced dysmetria |
| Brain MRI | Microcephaly with diffuse parenchymal | Microcephaly and global hypomyelination with |
| | volume loss, thin corpus callosum, and | progressive myelin loss. MR spectroscopy |
| | global hypomyelination, with progressive | was normal |
| | loss of myelination. MR spectroscopy | |
| | was normal | |
| Evoked potentials | Visual, somatosensory, and brainstem | At 3 years, visual and somatosensory evoked |
| | auditory evoked potentials at 4 years | potentials revealed delayed cortical |
| | revealed delayed cortical responses | responses, brainstem auditory evoked |
| | | potentials were normal |
| Electroencephalogram | Not indicated | Abnormal background but no epileptiform |
| | | discharges |
| Nerve conduction | Normal at 4 and 13 years | Normal at 12 years |
| studies and | | |
| Electromyography | | |

*Measured at 3 wk, growth parameters at birth not available, SD—standard deviation, N/A—not available

Clinical, quad-based exome sequencing of the proband revealed homozygosity for a novel missense variant in the gene EPRS1 [NM_004446.2: c.4444C>A; p.(Pro1482Thr)]. No alternative diagnosis was identified with whole-exome sequencing. Sibling 2 is homozygous for the variant, while both parents and their unaffected son are heterozygous. Immortalized lymphoblastoid cell lines (LCLs) were generated from affected siblings, carrier parents, and an unrelated control (Control 3), transformed with Epstein-Barr virus, and the EPRS1 variant was validated by Sanger sequencing (FIG. 1C).

### Dual Post-Transcriptional Mechanisms Drive Low EPRS1^{P1482T} Expression

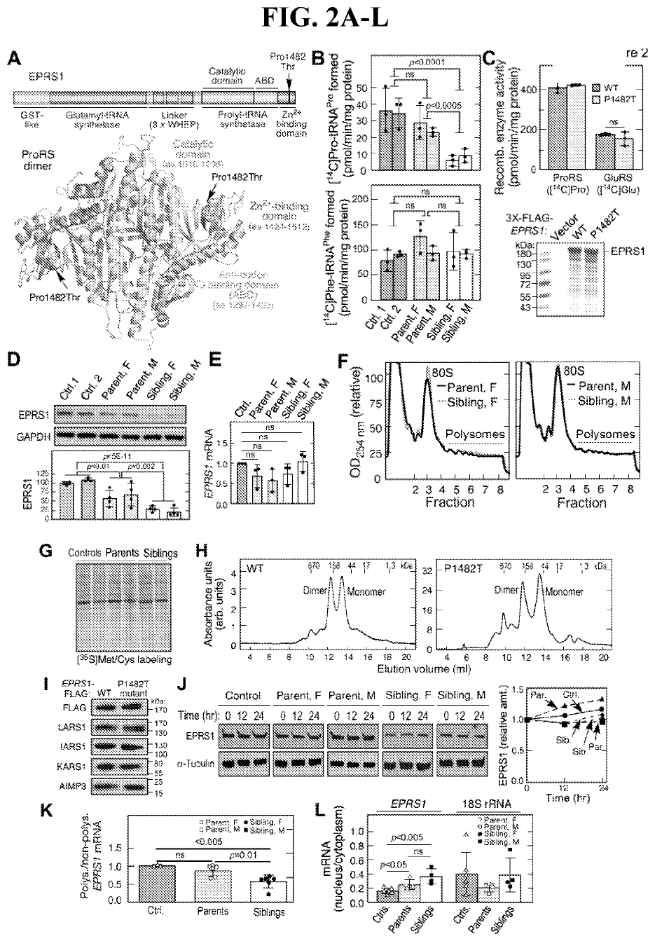

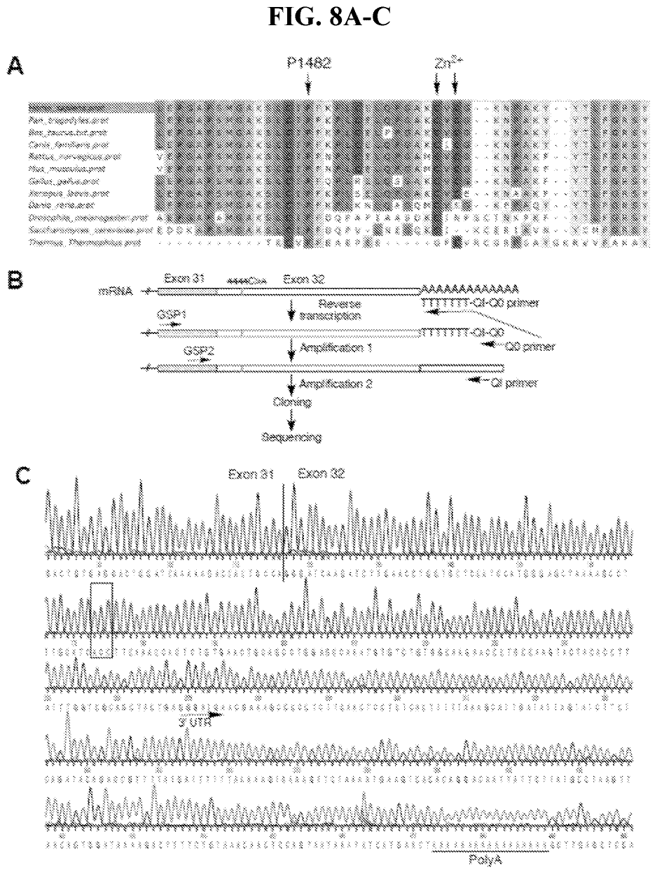

The Pro1482Thr substitution is in the Zn²*-binding domain of EPRS1, distant in sequence space from the catalytic and anti-codon binding domains (Vasu et al., 2021; Zhou et al., 2013) (FIG. 2A, top), but spatially near the intersection of the domains according to the X-ray structure of the human ProRS dimer (FIG. 2A, bottom) (Zhou et al., 2013). Pro¹⁴⁸² is in a highly conserved region and is present in all species investigated including S. cerevisiae, and possibly T. thermophilus (FIG. 8A). ProRS activity in LCL lysates was determined as charging of yeast tRNA with [¹⁴C]Pro (Halawani et al., 2018). Total ProRS charging activity by LCLs from affected siblings was about 20% of unaffected controls, and carrier parent LCLs exhibited intermediate activity (FIG. 2B, top). tRNA charging of [¹⁴C]Phe by FARS1 was comparable for all LCLs, indicating specificity of the inhibition of ProRS charging (FIG. 2B, bottom). Reduced cellular ProRS activity might reflect either decreased amount of EPRS1^{P1482T} or reduced specific charging activity. To test the latter mechanism, recombinant N-terminal, His-tagged wild-type (WT) and Pro1482Thr mutant EPRS1 were expressed and purified from HEK293T cells. Specific ProRS and GluRS charging activities were determined in vitro as incorporation of [¹⁴C]Pro and [¹⁴C]Glu, respectively, into yeast tRNA (Halawani et al., 2018). Specific activities of both catalytic domains were identical in WT and mutant EPRS1, suggesting diminished ProRS charging activity in the siblings is due to reduced EPRS1 amount, not specific activity (FIG. 2C). EPRS1 was determined in LCL lysates by immunoblot using antibody targeting the linker region. EPRS1 in the siblings was ~20% of that in unrelated controls bearing WT EPRS1; the carrier parents exhibited ~50-60% of control levels (FIG. 2D). Importantly, EPRS1 mRNA expression was nearly identical in all LCLs (FIG. 2E). Likewise, 3′-RACE analysis of EPRS1 mRNA in LCLs, from the next-to-last exon to the poly-A tail, and spanning the c.4444C>A variant in the terminal exon, indicated identical splicing and polyadenylation (FIG. 8 B, C).

These results indicate post-transcriptional regulation is responsible for reduced expression of sibling EPRS1. In view of the essential role of aaRSs in protein synthesis, the effect of the ~80% decrease in EPRS1 amount on total protein synthesis in LCLs from the siblings was determined by polysome profiling—a method that separates ribosome-rich, translating, polysomal mRNAs, from ribosome-free, non-translating mRNAs. Polysome profiles comparing protein synthesis in LCLs from the female sibling to the mother (FIG. 2F left) and male sibling to the father (FIG. 2F, right) are virtually identical; polysome profiles of unrelated controls were also identical (data not shown). Likewise, metabolic labeling with [³⁵S]Cys/Met confirmed that global protein synthesis, as shown by labeling nascent protein, is not reduced in sibling LCLs indicating an ~80% inhibition of EPRS1 expression is not injurious to cells (FIG. 2G), and consistent with undiminished charging activity of the EPRS^{P1482T} variant.

EPRS1 forms dimers via interactions of the ProRS domain (Zhou et al., 2013). The fate of EPRS1 protein could be influenced by its dimerization status, as well as by localization outside the MSC. Size fractionation of recombinant WT and Pro1482Thr ProRS showed similar extents of dimerization (FIG. 2H). To determine the influence of the EPRS1^{P1482T} substitution on residence in the MSC, FLAG-tagged, full-length WT and mutant EPRS1 cDNAs were transfected into HEK293T cells. EPRS1 was isolated from lysates with anti-FLAG resin, eluted with FLAG peptide, and subjected to immunoblot. WT and EPRS1^{P1482T} bind equally to the four MSC constituents tested, indicating normal MSC incorporation of the mutant (FIG. 2I). The stability of mutant EPRS1 was investigated directly. LCLs were treated with cycloheximide to block protein synthesis, and EPRS1 disappearance monitored by immunoblot. No loss was detected over a 24-h period in any LCL, indicating all EPRS1 forms are highly stable (FIG. 2J). The translation state of WT and mutant EPRS1 mRNA was explored by polysome profiling. An ~40% reduction in polysomal EPRS1 mRNA was observed in affected sibling LCLs, consistent with an important contribution of translation to reduced EPRS1 expression (FIG. 2K); however, the amount of reduction is less than that of the steady-state level of protein, suggesting additional mechanisms might be operative. A possible defect in nuclear export of newly transcribed c.4444C>A EPRS1 mRNA was explored by fractionation of LCL lysates into nuclear and cytoplasmic pools. The ratio of nuclear to cytoplasmic EPRS1 mRNA, as measured by primer/probe spanning the exon 3-4 junction, was ~2-fold higher in siblings compared to controls, and the parental level was intermediate, consistent with higher nuclear levels of c.4444C>A EPRS1 mRNA and diminished export (FIG. 2K). Together, these results indicate EPRS1 expression in sibling cells is reduced by dual post-transcriptional mechanisms.

Role of EPRS1 mRNA m⁶A Modification in Reduced Expression of Variant EPRS1

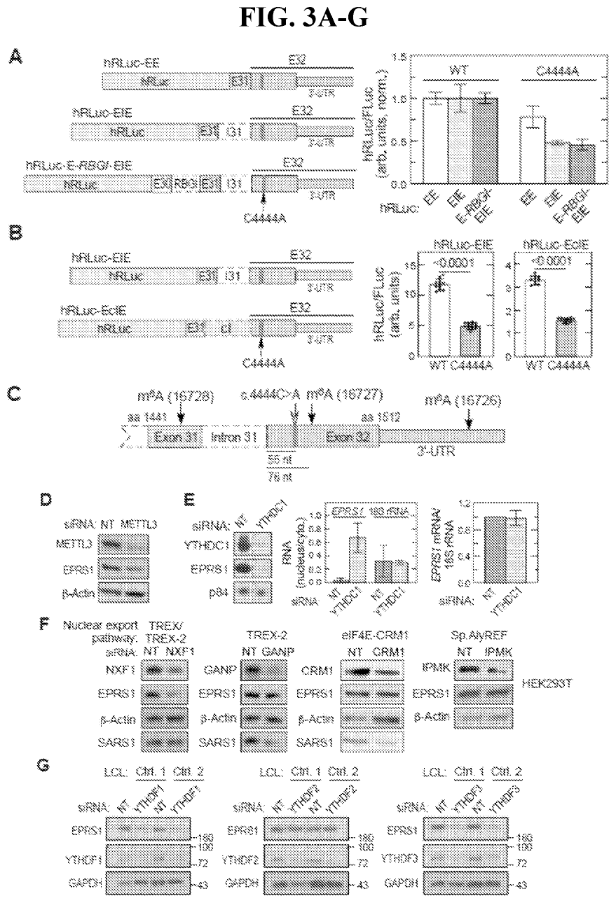

To investigate the mechanism underlying low expression of variant EPRS1, chimeric reporters were generated containing hRLuc upstream of the 3′-terminal region of EPRS1 mRNA surrounding the variant site, namely, exons 31 and 32, bearing either WT or c.4444C>A sites (hRLuc-EE) (FIG. 3A, left-top). In all reporters, the EPRS1 RNA sequence was in-frame with hRLuc, and without an intervening stop codon. Following transfection into HEK293T cells, expression of the hRLuc-EE reporter bearing the c.4444C>A variant was ~25% less than the WT—a lower level of inhibition than observed for endogenous EPRS1 mRNA (FIG. 3A, right). Because splicing facilitates nuclear mRNA export in mammalian cells (Valencia et al., 2008), we generated a reporter pair containing the intervening intron, 131 (hRLuc-EIE) (FIG. 3A, left-middle).

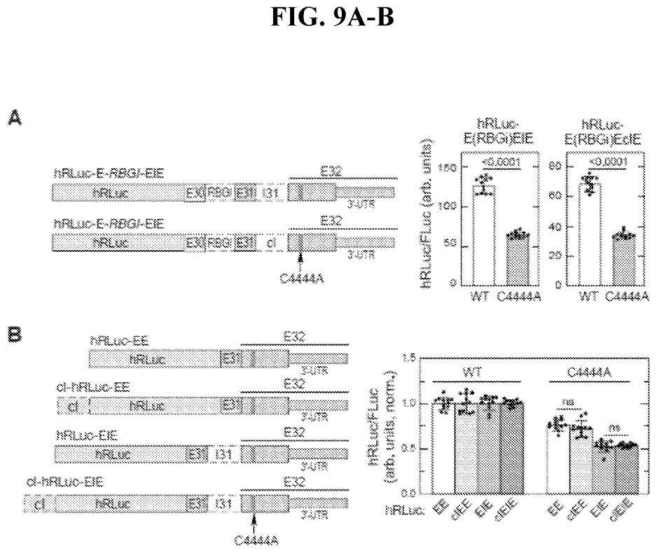

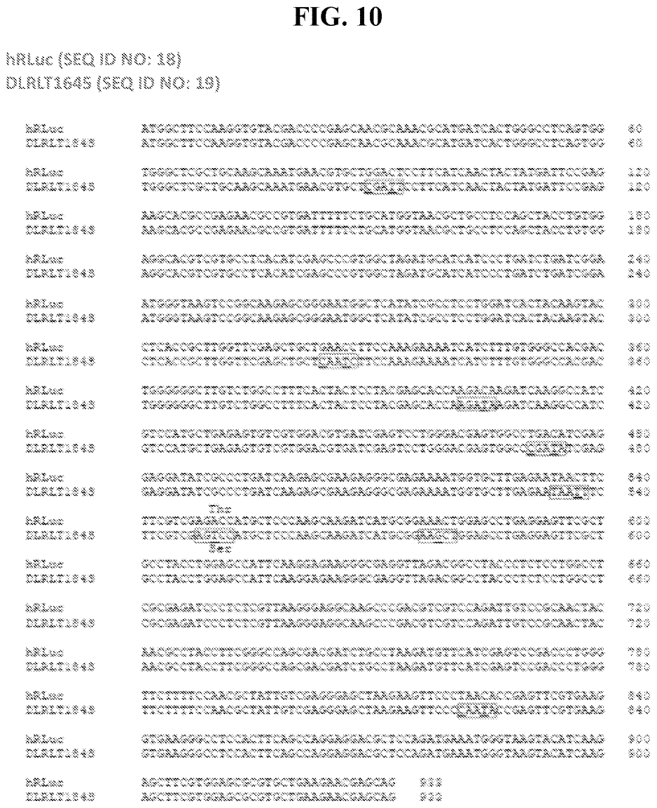

Inclusion of the intron induced a 50% decrease in expression of the reporter bearing the c.4444C>A variant. Introduction of a second upstream intron between exons 30 and 31 (hRLuc-E-RBGI-EIE, rabbit b-globin gene intron was used because the ~3 kb EPRS1 intron 31 contains a potential insertion sequence/transposon element with multiple inverted repeats) did not further reduce relative expression of the reporter bearing the c.4444C>A variant. To determine if sequences within the intervening intron influence expression, 131 was replaced by an unrelated chimeric intron, cI (FIG. 3B, left; S2A) (Lai et al., 2006). Although expression of the non-mutated reporter was reduced, expression of hRLuc-EcIE and hRLuc-E-RBGI-EcIE reporters bearing the c.4444C>A variant was inhibited to the same extent as reporters containing I31, i.e., hRLuc-EIE (FIG. 3B, right; S2A). An intron (e.g., cI) in the 5′UTR of hRLuc-EE reporter (FIG. 9B, left, top two schematics), does not synergize with the HLD-causing c.4444C>A variant (FIG. 9B, right). Synergy is evident only when an intervening intron (e.g., I31) is present between exons 31 and 32. (FIG. 9B, left, bottom two schematics), highlighting the specific role of this exon-exon junction. hRLuc-EIE was selected for subsequent reporter-based experiments.

The c.4444C>A variant site is in the terminal exon, exon 32, 55 nt downstream of the junction with exon 31 (FIG. 3C, left). Importantly, with respect to mRNA architecture, this is a “hotspot” for methylation of the N⁶-position of adenosine (m⁶A, N⁶-methyladenosine), the most abundant mRNA modification (Dominissini et al., 2012). Global analysis revealed more than 70% of all m⁶A residues in mRNAs are in the 3′-most exon, peaking just downstream of the exon start (Ke et al., 2015). Likewise, sequences recognized for m⁶A modification, i.e., DRACH (A/G/U-A/G-A-C-A/C/U) sequences, are enriched in terminal exons (Ke et al., 2015; Meyer et al., 2012). m⁶A modification and its cellular consequences are dictated by sequence-specific writers, erasers, and readers (Patil et al., 2018). Importantly, occupancy of m⁶A-modified sites determines both nuclear export and translation, as well as mRNA stability (Lesbirel et al., 2018; Meyer et al., 2012; Roundtree et al., 2017; Zaccara et al., 2019). Human EPRS1 mRNA exhibited three experimentally confirmed m⁶A sites in the region near the c.4444C>A site in m⁶A-Atlas: an upstream site in exon 31 (16728), and two downstream sites in terminal exon 32—one in the coding region (16727), and another in the 3′-UTR (16726) (FIG. 3C, left) (Tang et al., 2021). The potential role of m⁶A in determining EPRS1 expression was investigated by knockdown of METTL3, the catalytic component of the principal m⁶A writer complex (Lesbirel et al., 2018). METTL3 knockdown in HEK293T cells markedly reduced EPRS1 expression (FIG. 3D). YTHDC1 is a member of a family YTH domain-containing proteins that are m⁶A readers that regulate mRNA stability and translation, as well as nuclear export (Patil et al., 2018); specifically, YTHDC1 is a nuclear reader of m⁶A-modified mRNA that regulates nuclear mRNA export (Lesbirel et al., 2018), siRNA-mediated knockdown of YTHDC1 in control LCLs inhibited EPRS1 expression, implicating m⁶A modification in export of EPRS1 mRNA (FIG. 3E, left). YTHDC1 knockdown dramatically increased nuclear retention of EPRS1 mRNA as shown by cell fractionation and RT-qPCR (FIG. 3F, center), but did not influence total EPRS1 mRNA (FIG. 3F, right).

Nuclear mRNAs are packaged into messenger ribonucleoprotein complexes and exported from the nucleus via a family of nuclear pore complexes embedded in the nuclear envelope (Carmody and Wente, 2009). Importantly, the transcription-export complex (TREX), in association with YTHDC1, has primary responsibility for nuclear export of m⁶A-modified mRNAs (Lesbirel et al., 2018). To determine the nuclear RNA exporter responsible for EPRS1 mRNA export, specific constituents of nuclear exporters were subjected to siRNA-mediated knockdown (Wickramasinghe and Laskey, 2015). Knockdown of NXF1 (nuclear RNA export factor 1), an integral component of TREX, markedly inhibited expression of EPRS1; whereas seryl-tRNA synthetase (SARS1) expression was not inhibited (FIG. 3G, left panel). Knockdown of GANP (germinal center-associated nuclear protein) of the transcription-export complex-2 (TREX2) pathway, CRM1 (chromosomal maintenance 1) of the eIF4E-CRM1 pathway, or IPMK (inositol polyphosphate multikinase) of a specialized AlyREF pathway, did not inhibit EPRS1 expression, but interestingly, the first two inhibited expression of SARS1 (FIG. 3G, right-most 3 panels). The results implicate the METTL3-YTHDC1-TREX pathway as the nuclear export pathway utilized by m⁶A-modified EPRS1 mRNA, and suggest that the c.4444C>A variant negatively influences the function of one or more pathway constituents.

The YTHDF series of cytoplasmic m⁶A readers (YTHDF1/2/3) facilitate translation and mRNA stability; YTHDF1 and YTHDF3 regulate translation and YTHDF2 and YTHDF3 regulate mRNA stability. However, there is uncertainty on the relative importance of these reader functions (Shulman and Stern-Ginossar, 2020). Knockdown of YTHDF1 and YTHDF3 in control LCLs markedly reduced EPRS1 expression, whereas knockdown of YTHDF2 was without effect (FIG. 3G). Thus, optimal EPRS1 expression in healthy cells requires YTHFDC1-mediated nuclear export via NXF1, followed by YTHDF1/3-mediated translation.

Identification of EPRS1 mRNA m⁶A Sites Influenced by the c.4444C>A Variant

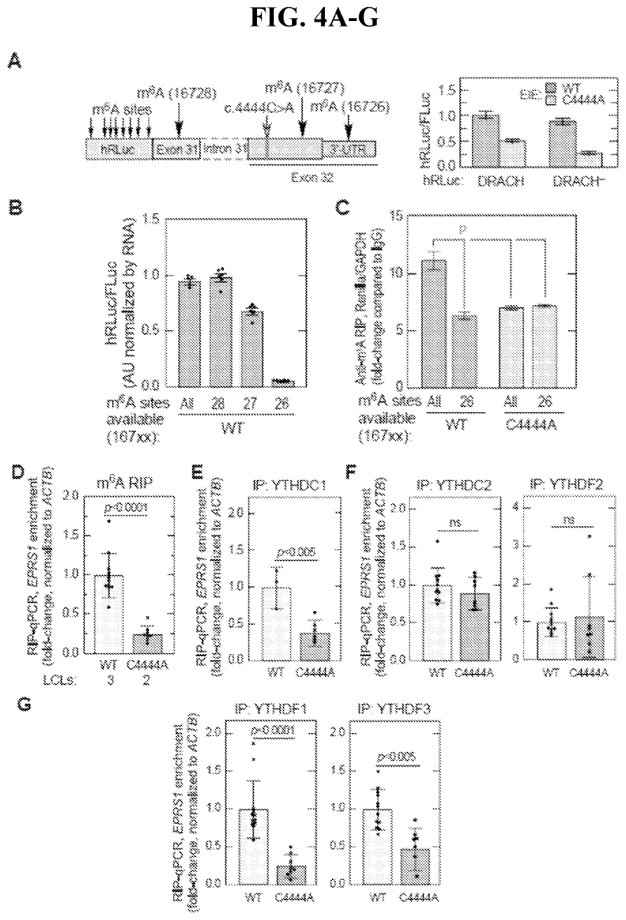

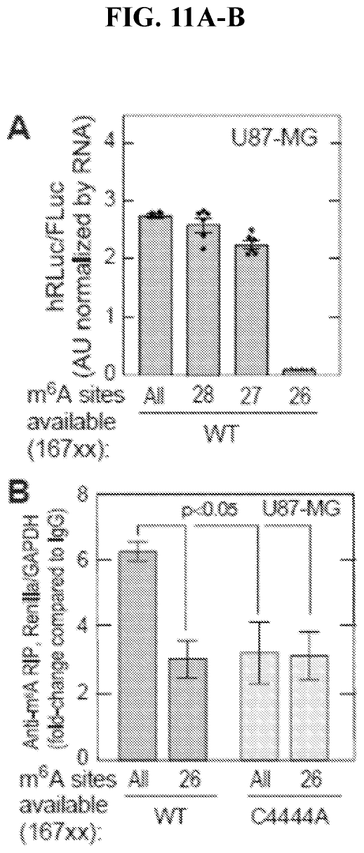

To facilitate investigation of m⁶A modification of the EPRS1 reporter, background m⁶A modification of hRLuc RNA was reduced by generating constructs in which the eight DRACH sites in hRLuc were nullified by synonymous mutation, except for an obligate Thr184Ser mutation (FIGS. 4A, left and 10). Following transfection into HEK293T cells, activity of the DRACH-containing WT construct was slightly lower than the DRACH⁺ reporter, possibly due to the non-synonymous substitution near the active site (Loening et al., 2007); however, reduced expression by c.4444C>A variant was retained, or possibly improved (FIG. 4A, right), enabling an assay for m⁶A modification. To determine the specific m⁶A site (or sites) contributing to EPRS1 expression, the three experimentally confirmed EPRS1 m⁶A sites were pairwise inactivated by mutation in the DRACH⁻ hRLuc construct. Sites 16727 and 16728 were disrupted by synonymous mutations, while 3′UTR-site 16726 was disrupted by minimally altering the minimum energy-predicted RNA structure. In the context of the WT C4444 sequence, simultaneous disruption of the 16727 and 16728 sites (thus permit-ting modification of the 16726 site only) almost completely blocked hRLuc expression, but mutation of the other pairs, 16726/16728 and 16726/16727, did not reduce expression (FIG. 4B). This result indicates that m⁶A modification of either 16727 or 16728 (16728 is position A4355, and 16727 is position A4464, in the EPRS1 mRNA), the sites flanking the c.4444C>A site, are sufficient to induce hRLuc expression, but mutation of both sites prevents expression. Virtually identical results were observed in the U87-MG glioblastoma cell line indicating the mechanism is cell type-independent (FIG. 11A).

The role of the c.4444C>A variant in m⁶A modification was directly assessed by RNA-immunoprecipitation (RIP) in which the DRACH⁻ hRLuc reporter was transfected into HEK293T cells and subjected to immunoprecipitation with anti-m⁶A antibody, followed by RT-qPCR using primers for hRLuc. Elimination of both 16727 and 16728 sites reduced m⁶A modification of the DRACH⁻ hRLuc reporter in HEK293T cells (FIG. 4C) and in U87-MG cells (FIG. 111B). Likewise, mutation of the C4444 site inhibited m⁶A modification to about the same extent as m⁶A site modification. This experiment validates m⁶A modification of the EPRS1 mRNA reporter, and shows that the c.4444C>A variant inhibits m⁶A modification of critical sites responsible for EPRS1 expression.

The specific m⁶A-related defect that reduces expression of variant EPRS1 was investigated in patient LCLs. In a methylated RNA immunoprecipitation (meRIP)-qPCR approach, anti-m⁶A antibody pulldown of c.4444C>A variant EPRS1 mRNA was reduced by ~75% compared to controls, confirming reduced m⁶A modification of endogenous EPRS1 mRNA (FIG. 4D). Variant EPRS1 mRNA exhibited reduced interaction with YTHDC1, the m⁶A reader that directs mRNA nuclear export, confirming the reporter experiments (FIG. 4E). YTHDC2 and YTHDF2 are cytoplasmic m⁶A readers that regulate mRNA stability (Du et al., 2016; Kretschmer et al., 2018); neither protein exhibited differential binding to variant EPRS1 mRNA (FIG. 4F). YTHDF1 and YTHDF3, facilitate translation of bound mRNAs (Chang et al., 2020; Wang et al., 2015). YTHDF3 tunes the translation-activating role of YTHDF1 on m⁶A-modified RNA, and can influence mRNA stability in conjunction with YTHDF2 (Shi et al., 2017). Importantly, both YTHDF1 and YTHDF3 binding to variant EPRS1 mRNA was diminished compared to wild-type mRNA (FIG. 4G). Remarkably, a single point mutation near the stop codon of EPRS1 mRNA reduces m⁶A modification at two sites, inhibits binding of three YTH domain family proteins, and consequently reduces both mRNA nuclear export and cytoplasmic translation.

Reduced m⁶A Site Availability in Predicted c.4444C>a Variant-Specific mRNA Structure

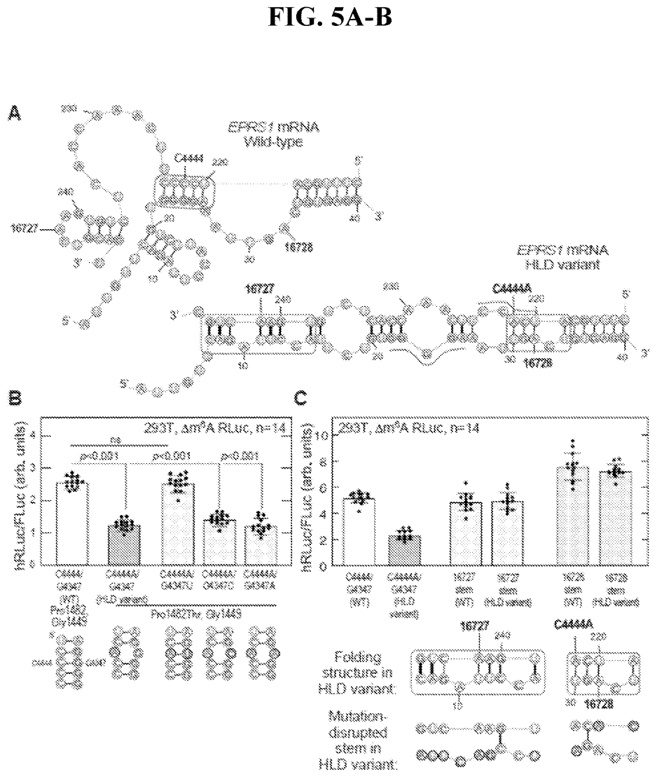

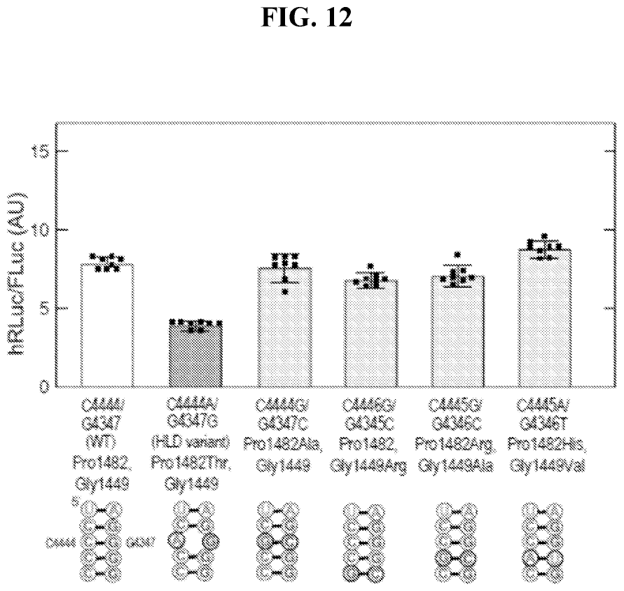

mRNAs can exhibit partially unfolded RNA structures during translation, and m⁶A impacts RNA structure (Liu et al., 2015; Spitale et al., 2015). Highlighting a case where local RNA structure can impact m⁶A modification, we propose that the conformation of WT EPRS1 mRNA permits m⁶A modification in terminal exons 31-32, but C-to-A substitution at nt 4444 induces a conformational switch that reduces or prevents modification. Minimum energy folding (RNAStructure 6.4) (Reuter and Mathews, 2010) of the local WT sequence, i.e., exon 31 and exon 32 up to the stop codon without the intervening intron, indicates C4444 is in a 5-bp stem including four sequential G-C base pairs (FIG. 5A, top); inclusion of the intron does not alter the folding structure in this region. The stem potentially drives a conformation in which the critical modified adenosine residues (16727, 16728) are unpaired within loop structures, consistent with susceptibility to m⁶A modification (Meiser et al., 2020). The folding analysis indicates the c.4444C>A variant disturbs the 5-bp stem, and the alternative structure is stabilized by two separate stems encompassing the m⁶A sites surrounding the variant site (FIG. 5A, bottom). The critical adenosine residues are within the base-paired stems, and thus less susceptible to m⁶A modification by the METTL3 complex (Meiser et al., 2020). The role of the 5-bp stem on reporter expression was explored by mutagenesis in the DRACH⁻hRLuc background. Mutation of C4444 to A, i.e., generation of the patient variant, reduced hRLuc expression by about half, but restoration of the predicted stem by G4347U mutation in the opposing strand to generate an A-U base pair restored reporter expression (FIG. 5B). Mutation of G4347 to non-complementary nts C or A did not restore reporter activity, supporting the predicted local structure and the critical role of the 5-bp stem. The complementary mutations retained the amino acids encoded by the c.4444C>A (p.Pro1482Thr) reporter, indicating that amino acid sequence in the HLD variant is not responsible for the reduced expression. Three stem G-C pairs were reversed to C-G (including one encompassing the variant), one also was exchanged for an A-U pair, and all exhibited WT reporter activity, providing further evidence for the predicted stem (FIG. 12). In an orthogonal approach, masking of the m⁶A sites by the putative stems in the variant mRNA was tested by mutations designed to disrupt the stems and expose cryptic m⁶A sites. Disruption of the stem surrounding the 16727 m⁶A site, by a series of synonymous and non-synonymous mutations (Gln1444Ile, Ile1445Gln, syn.Pro1446Pro) that minimally altered primary protein sequence, restored expression of the reporter bearing the C4444A mutation (FIG. 5C). Likewise, mutation of residues in the stem surrounding the 16728 m⁶A site (syn.Ile1451Ile, Ile1481His), restored, and possibly exacerbated, reporter expression. These results are consistent with the previous finding that m⁶A modification of a single site is sufficient for EPRS1 expression. Together, the effects of the mutations suggest a mechanism in which the c.4444C>A variant reduces accessibility of the m⁶A site to the methyltransferase, and that the predicted RNA structure, not linear sequence, is the critical determinant of reporter expression.

mRNA-Targeted Rescue of Defective Expression of c.4444C>a Variant

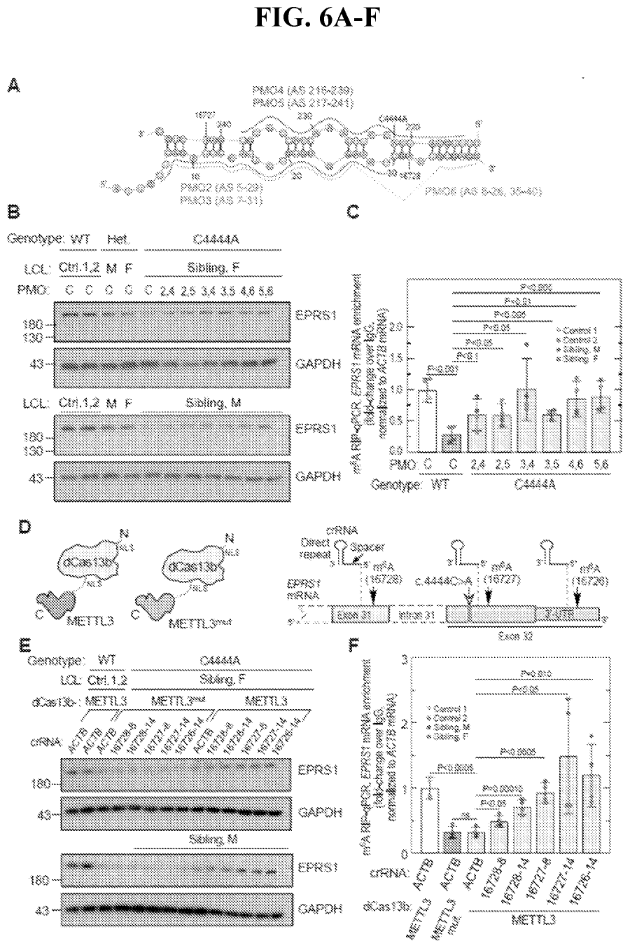

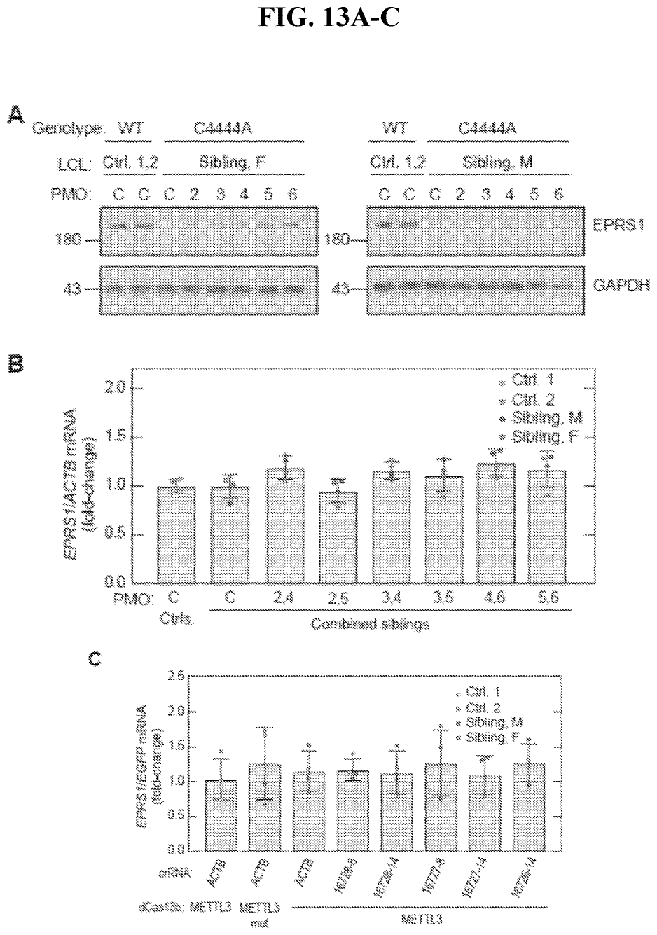

The structure-based inhibition of m⁶A modification suggests that non-genetic intervention might also increase availability and modification of m⁶A sites. Antisense phosphorodiamidate morpholine oligonucleotides (PMOs) were generated to target both strands in the region of the variant mRNA containing the m⁶A sites (FIG. 6A, top). PMOs were applied to patient LCLs, and EPRS1 in lysates determined by immunoblot. All PMOs tested induced EPRS1 expression compared to a control PMO; induced expression by all PMOs was higher in the female patient compared to the male (FIG. 13A). To increase expression, PMOs were added pairwise, in combinations targeting both strands of the c.4444C>A variant-specific structure. The expression was increased, particularly with the PMO5/PMO6 pair to nearly the level in the heterozygous parental LCLs (FIG. 6B). Increased protein expression was not accompanied by increased EPRS1 mRNA consistent with post-transcriptional regulation (FIG. 13A), Enhanced m⁶A modification of EPRS1 mRNA by PMOs was shown by meRIP-qPCR (FIG. 6C).

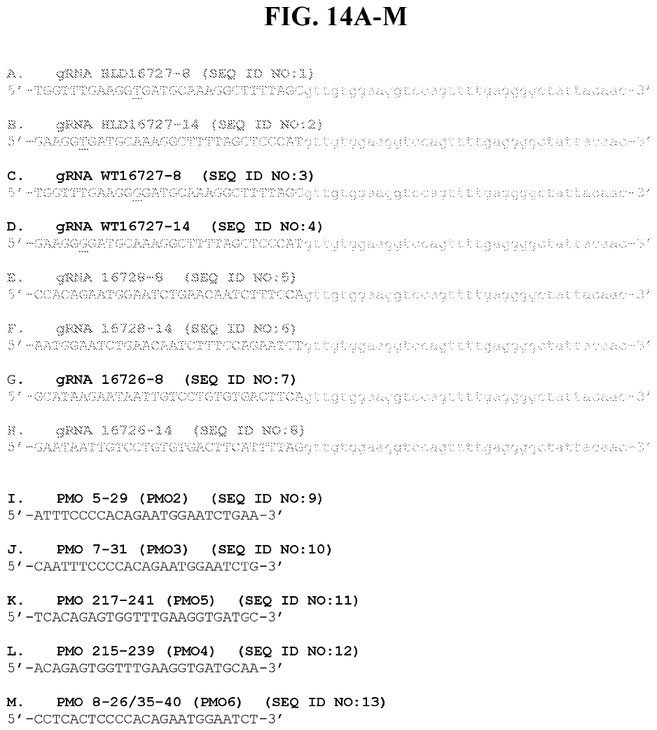

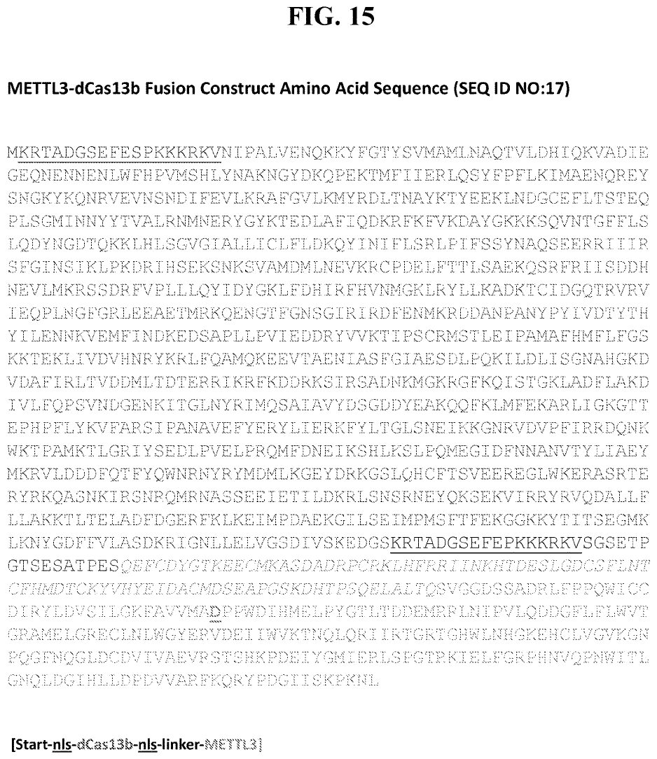

In a second approach, rescue of defective m⁶A-modification in variant EPRS1 mRNA was attempted using targeted RNA methylation (TRM) (Wilson et al., 2020). LCLs were nucleofected with catalytically-dead Cas13b (dCas13b) bearing nuclear localization signals (NLS), and fused to truncated METTL3 methyltransferase; inactive METTL3 mutant (METTL3^{mut}) served as a specificity control (Sun et al., 2022; Wilson et al., 2020) (FIG. 6D. left). The cells were nucleofected with CRISPR RNAs (crRNAs) (see FIG. 14) complementary to regions upstream of the m⁶A site and targeted by the dCas13b-METTL3 chimera. crRNAs were generated to target protospacer sequences 8 or 14 nt upstream of the three m⁶A sites in exons 31 and 32 (FIG. 6D, right); crRNA targeting b-actin mRNA (ACTB) served as a control. dCas13b-METTL3 chimeras and crRNAs were nucleofected into control LCLs and sibling LCLs, and cells grown for 4 days. Nucleofection of dCas13b-METTL3 with crRNAs targeting each m⁶A site showed at least partial rescue of EPRS1; crRNAs targeting the two 3′-most sites were most effective in restoring in both patient LCLs approaching that of control LCLs. As a control, crRNAs co-transfected with dCas13b-Mettl3^{mut} were ineffective (FIG. 6E), demonstrating m⁶A writer-dependent rescue. These results provide direct evidence for the role of deficient m⁶A modification in the expression of c.4444C>A variant EPRS1 mRNA. In parallel experiments, enhanced m⁶A modification of c.4444C>A variant EPRS1 mRNA roughly corresponding to the stimulation of expression was shown by meRIP-qPCR (FIG. 6F). Together, these results support a pathologic mechanism of a variant-induced masking of sequence-distal m⁶A sites that prevents access by the methyl-transferase, as well as potential therapeutic modalities.

There is emerging interest in the role of m⁶A modification of mRNA in human pathology (Jiang et al., 2021; Yang et al., 2020). In many cases, the expression of an enzyme constituent of the m⁶A-modification pathway is altered. For example, FTO (fat mass and obesity-associated protein) the major m⁶A eraser, i.e., demethylase, is highly expressed in multiple acute myeloid leukemias, thereby enhancing oncogene-mediated cell transformation and leukemogenesis (Li et al., 2017). However, little is known about disease-causing pathogenic variants in these pathway enzymes. An exception is the pathogenic variant in FTO in a consanguineous multiplex family of Palestinian Arabs responsible for an autosomal-recessive lethal syndrome, and death before 30 months (Boissel et al., 2009). The variant inactivated the DNA demethylation activity, and although it was not shown, it's likely that demethylation of m⁶-modified mRNA was likewise inactive. Also, homozygous pathogenic variants in the m⁶A reader YTHDC2 in three women are associated with early-onset primary ovarian insufficiency (McGlacken-Byrne et al., 2022), a clinical feature also seen in Sibling 2.

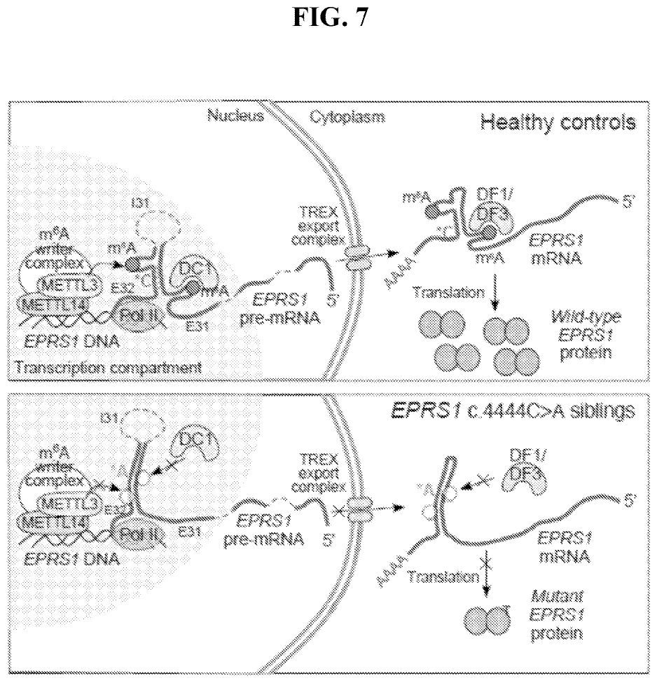

Despite the prevalence of m⁶A-modified mRNAs, few pathogenic variants in the target sites have been associated with disease. Two variants that generate pathologic m⁶A sites have been reported, both involving tumor suppressors. A G>A variant of the tumor suppressor p53 introduces an Arg273His missense substitution that promotes m⁶A modification of the mutant codon and increases expression, possibly by enhanced pre-mRNA splicing; the variant exhibits enhanced drug resistance (Uddin et al., 2019). In a second study, a G>A variant of ANKLE1, a suppressor of colorectal cancer (CRC), induced m⁶A modification, increasing ANKLE1 expression and reducing CRC risk (Tian et al., 2020). Our work uniquely reveals a pathologic transcript variant that prevents, rather than induces, m⁶A modification at well-defined sites. More importantly, the patient C4444A EPRS1 mRNA variant is not at the critical m⁶A modification sites, nor within the surrounding consensus sequences, but at a distant residue. The mutation reduces m⁶A modification, likely by an alteration of local mRNA conformation that reduces accessibility of otherwise functional m⁶A sites. Also notable is the finding that a single point mutation in the open reading frame of EPRS1 mRNA reduces both nuclear export and translation in patient LCLs, resulting in pathologically low levels of EPRS1 protein (FIG. 7). The requirement for m⁶A modification of wild-type EPRS1 mRNA for efficient expression of protein was unexpected, particularly in view of the observation that most mRNAs encoding “housekeeping” proteins, for example, ribosomal proteins, are de-enriched in m⁶A (Zaccara et al., 2019). In addition to defective m⁶A modification of mutant EPRS1 mRNA in patient-derived LCLs, defective m⁶A modification was observed in mutation-bearing reporters in embryonic kidney-derived HEK293T cells and glioma-derived U87-MG cells. The apparent lack of cell-type specificity is consistent with observations by others that m⁶A sites are generally constitutive with similar distributions in tissues and cell lines (Zaccara et al., 2019). Likewise, the m⁶A enzymes, including writers and readers are present in most tissues including major brain cell classes, such as neurons and neuroglia, the cell types likely to be adversely affected by dysregulated expression in HLD patients (Yang et al., 2020).

Four other patients with distinct bi-allelic pathogenic variants in EPRS1 exhibiting childhood-onset HLD15 have been reported (Mendes et al., 2018). Two are homozygous for missense variants in the ProRS catalytic core domain; the other two are compound heterozygous, characterized by one allele with a missense variant in the ProRS catalytic core and a premature stop codon in the other allele. The best studied of these mutant forms is the c.3344C>G variant that encodes a p.Pro1115Arg substitution in the catalytic core domain predicted to influence specific activity. Indeed, an ~30% decrease in charging activity by recombinant ProRS was shown as well as a 40% decrease in EPRS1 protein in patient fibroblasts. In vitro charging activity in lymphoblast lysates was determined in one of the patients and showed an ~70% reduction compared to controls. These results, combined with our own, suggest a mechanism in which diminished total charging activity—whether due to protein amount or specific activity, or both—contributes to the pathologic defect.

The mechanistic link between reduced EPRS1 and CNS hypomyelination remains elusive. The obligate role of EPRS1, like all aaRSs, in interpretation of the genetic code during translation suggests that reduced protein synthesis is a major contributor to pathology. The undiminished total protein synthesis in sibling LCLs argues against this mechanism. However, low levels of EPRS1 might specifically inhibit protein synthesis in critical cells particularly sensitive to tRNA charging activity, e.g., in myelinating oligodendrocytes. Consistent with tissue-selective responses to aminoacylation defects, fibroblasts from patients with compound heterozygous mutations in the GluRS region of EPRS1 exhibited normal growth rates despite severely compromised tRNA charging activity of recombinant protein in vitro (Jin et al., 2023). Although genetic defects in two other cytoplasmic aaRSs, namely, DARS1 and RARS1, also cause HLD (Choquet et al., 2017; Taft et al., 2013; Wolf et al., 2014), defects in at least ten cytoplasmic aaRSs cause distinct neurologic disorders including encephalopathy, microcephaly, as well as the peripheral nervous system disease, CMT (Mendes et al., 2018; van der Knaap and Bugiani, 2017), suggesting that aaRS inhibition of protein synthesis is unlikely to be the principal etiology underlying HLD. Alternatively, the defect might be due a defective noncanonical function of EPRS1, possibly a CNS-specific function, unrelated to protein synthesis (Arif et al., 2017; Lee et al., 2022; Lee et al., 2016; Sampath et al., 2004). A possible insight is the observation that three of the variant genes that cause HLD, i.e., EPRS1, DARS1, and RARS1, encode constituents of the MSC which houses nine of the twenty cytoplasmic aaRSs. In addition, variants in two non-aaRS MSC constituents, i.e., AIMP1 and AIMP2, also cause leukodystrophy, although the former is likely to be secondary to a peripheral neurodegenerative disorder (Boespflug-Tanguy et al., 2011; Feinstein et al., 2010; Mazaheri et al., 2022; Shukla et al., 2018). Thus, a dysfunctional MSC might contribute to HLD pathology. However; elucidation of this mechanism is hampered by the current lack of understanding of the critical function(s) of the MSC which appears to be unrelated to efficiency of protein synthesis (Cui et al., 2021).

Improved understanding of the pathophysiology of leukodystrophies, aided by state-of-the-art molecular technologies, has begun to guide potential therapeutic approaches, including drug design and gene therapy (van der Knaap et al., 2019). For example, an RNA-based therapeutic targeting of proteolipid protein 1 (PLP1), the gene defective in PMD, has been investigated. Administration of antisense oligonucleotides targeting PLP1 restored myelination and motor function in the jimpy (Plp1(jp)) mouse model of severe PMD (Elitt et al., 2020). Likewise, a morpholino was used to correct aberrant splicing in a mouse bearing a PLP1 exon 3 variant that in humans causes PMD, spastic paraplegia 2, and hypomyelination of early myelinating structures (Tantzer et al., 2018). Lastly, administration of a locked nucleic acid antisense oligonucleotide targeting N-acetyltransferase 8-like (Nat81), the enzyme that generates N-acetyl-L-aspartate, reverses ataxia in a mouse model of Canavan disease, a vacuolar leukodystrophy (Hull et al., 2020). In experiments described here, the mechanism of inhibition has significant implications regarding potential treatment to restore EPRS1 mRNA processing. Diminished expression of the variant EPRS1 results from a single well-defined molecular event that suppresses both nuclear export and translation, namely, defective m⁶A modification of the transcript. Rationally-designed, antisense PMOs were deployed to disrupt the predicted, variant-specific RNA secondary structure to reveal masked m⁶A sites. Antisense PMOs, e.g., eteplirsen and golodirsen, are FDA-approved for clinical management of Duchenne muscular dystrophy (Frank et al., 2020; Mendell et al., 2013) by altering splicing of mutant mRNA. Alternatively, tethering catalytically-inactive dCas13 to m⁶A writers, erasers, and readers for programmable, site-specific introduction of m⁶A has the potential to transform fundamental studies of RNA methylation, as well as clinical application (Sun et al., 2022). Taking advantage of a targeted RNA methylation system (Wilson et al., 2020), nucleus-localized dCas13b fused with truncated METTL3 methyltransferase was co-transfected with guide RNAs cognate to sites upstream of m⁶A sites in the two terminal exons of EPRS1 mRNA. Highly effective restoration of EPRS1 expression in patient LCLs expands the therapeutic toolkit to ameliorate low EPRS1 expression associated with the c.4444C>A variant.

Single nucleotide polymorphisms (SNPs) and variants (SNVs) alter protein expression and function by multiple mechanisms—foremost is alteration of protein sequence and structure by non-synonymous mutation. Nonetheless, synonymous mutations also can dictate profound functional consequences by altering mRNA splicing, transcription and translation factor binding, mRNA stability, and translation rate (Hunt et al., 2014). Recently, mRNA m⁶A modifications have been recognized as pathological SNP targets, either by altering m⁶A writer and reader activities, or by inducing gain- or loss-of-function mutation of m⁶A sites, termed m⁶A-SNPs (Niu and Zhou, 2022).

### Determination of Total Protein Synthesis by LCLs

LCLs (0.5×10⁶ cells) were pre-incubated in methionine-free RPMI medium (Invitrogen) with dialyzed FBS (ThermoFisher) for 30 min, followed by addition of [³⁵S]Met/Cys (0.01 mCi, Perkin-Elmer) for 15 min at 37 °C. Labeled cells were lysed in RIPA buffer (Sigma) with protease and phosphatase inhibitors. Lysate from equal numbers of cells were was resolved by SDS-PAGE. Gel was fixed in 40% methanol, 10% acetic acid, and processed by autoradiography.

### Purification of Recombinant ProRS and Determination of Oligomeric State

Wild-type or P1482T mutant ProRS fragment of EPRS1 (aa 930-1512) was subcloned in pTRC-HisB (Invitrogen) for N-terminal 6×-His tagging, and sequence verified. Recombinant protein was expressed in BL21 Codon-plus (DE3)RIPL (Agilent) strain as described (Arif et al., 2017a; Halawani et al., 2018). Briefly, protein was induced with isopropyl β-D-1-thiogalactopyranoside (200 mM) at 37 °C, and cells harvested by centrifugation 4-6 hr post-induction. The pellet was resuspended in purification buffer containing 50 mM Tris-HCl, pH 8.0, 100 mM NaCl, 10% glycerol, 1 mg/ml lysozyme, protease inhibitors, and 10 mM imidazole, and sonicated on ice for 20 min. Lysate was cleared by centrifugation at 26,000×g for 45 min, and purified using HisTrap HP column (GE Life Sciences, Pittsburgh, PA). Protein oligomeric state was determined using a Superdex 200 size-exclusion column (GE Life Sciences) pre-calibrated with Bio-Rad gel-filtration standards (Bio-Rad) in purification buffer using an Akta purifier system (GE Life Sciences). ProRS aminoacylation activity was confirmed as described (Halawani et al., 2018).

### Western Blot Analysis and Antibodies

Samples harvested from cells or by immunoprecipitation and elution were mixed with RIPA buffer (Sigma), and subjected to SDS-PAGE. Antibodies against EPRS1, KARS1, LARS1, IARS1, AIMP3, SARS1, p84, METTL3, YTHDC1, YTHDC2, YTHDF1, YTHDF2, YTHDF3, GANP, NXF1, CRM1, IPMK, β-actin-HRP, FLAG, GAPDH-HRP and α-tubulin-HRP were used in immunoblot analysis (refer to Key Resources Table for details on sources, dilutions, and validations).

mRNA Determination in Nuclear and Cytoplasmic Fraction

LCLs (~5 million) were spun at 200 g for 5 min, pellet washed once with PBS, and re-suspended. Cells were spun again at 200 g for 5 min, and pellet subjected to cytoplasmic and nuclear RNA fractionation using PARIS kit (Life Technologies). RNA fractions were collected in 60 ml of kit elution solution and treated with Turbo DNase (Life Technologies) per manufacturer's protocol in 70 ml reaction volume. Following inactivation, 4 ml of RNA solution was used for RT-qPCR using AgPath-ID One Step kit (Life Technologies) in 10 ml reaction volume. For determination of nuclear and cytoplasmic RNA content in YTHDC1 knockdown experiments, ~2 million control LCLs were nucleoporated with YTHDC1 siRNA or Non-Targeting siRNA #1 (Silencer Select, Invitrogen) using Nucleofector II (Lonza) and Cell Line Nucleofector Kit V (Lonza) using program X005. After 48 hr, RNA was isolated and subjected to RT-qPCR as above.

### Cell Transfection

For plasmid DNAs, HEK293T and U87-MG cells were transfected with lipofectamine 2000 for 24-72 h. For siRNAs, HEK293T cells were transfected with lipofectamine RNAiMAX and 50-100 nM targeting siRNAs or Non-Targeting siRNA #1 (Silencer Select, Invitrogen) for 72 h. Transfection mixes were made in OptiMEM-I and added to cells in fresh growth medium.

### Luciferase Assay

HEK293T and U87-MG cells were co-transfected with various hRuc reporter and FLuc control plasmids using Lipofectamine 2000 for 24 h. Following cell lysis with Passive Lysis Buffer (Promega) for 20 min according to the manufacturer's protocol, Renilla and firefly luciferase activities were determined using Renilla Glo and Luciferase Assay Systems (Promega), respectively, using a Perkin-Elmer Victor³ luminometer or SpectraMax i3X multimode microplate reader.

Immunoprecipitation of m⁶A-Modified RNA (MeRIP) and RT-qPCR

Total RNA was isolated from HEK 293T and U87-MG cells transfected with DRACH-less hRLuc plasmids, or from LCLs untreated or treated with PMOs or TRM editors. Per 30 mg total RNA to be immunoprecipitated, 1 mg of anti-m⁶A antibody (Synaptic Systems) or rabbit IgG isotype control (Cell Signaling) and 5 ml protein A/G Dynabeads (Invitrogen) were incubated in binding buffer containing 50 mM Tris pH 7.6, 50 mM NaCl, 1 mM DTT, and 100 U/ml RnaseOUT for 30 min at room temperature, followed by 30 min at 4 °C with rotation. The bead-antibody slurry was washed twice and resuspended in ice-cold binding buffer. 10-60 mg of total RNA was used for immunoprecipitation with bead-antibody slurry in a final volume of 200-400 ml of ice-cold binding buffer. Tubes were rotated at 4 °C for 2 hr, followed by three washes in ice-cold binding buffer on a chilled magnetic separator. Washed beads were resuspended in Trizol, vortexed 20 sec, and stored at −80 °C. Extraction of m⁶A-modified RNA was done using RNeasy Mini kit (Qiagen) with on-column Dnase-I digestion. Equal volumes of eluted RNA were used in one-step RT-qPCR with Ag-Path ID Kit. For reporter-transfected cells, fold-change in ΔΔCₜ values was obtained for Renilla mRNA (refer to Key Resources Table for details of probe-primer sets) with GAPDH mRNA as control, from anti-m⁶A-IP compared to IgG-IP. For LCLs, fold-change in ΔΔCₜ values was obtained for EPRS1 mRNA with ACTB mRNA as control, from anti-m⁶A-IP compared to IgG-IP for sibling LCLs. Similarly derived values from control LCLs were used as baseline to calculate fold-change of EPRS1 mRNA immunoprecipitated with anti-m⁶A antibody.

### RNA Immunoprecipitation (RIP) and RT-qPCR

LCLs were harvested by centrifugation at 200 g for 5 min and washed once with PBS. Pellets were lysed in ~300 □l IP buffer (20 mM Tris pH 7.5, 100 mM KCl, 5 mM MgCl₂, 10 mM sodium orthovanadate, 0.2% Triton X-100, 1 mM DTT, 1× Halt protease and phosphatase inhibitors (Thermo), and 100 U/ml RNaseOUT) per 10⁶ cells. Cells were lysed by gently pipetting the pellet 10 times and then mixing in an end-to-end rocker at 4 °C for ~45 min. The supernatant was collected after centrifugation at 13,000 g for 5 min at 4 °C. Cell extracts were diluted to halve Triton X-100 concentration using detergent-free IP buffer and incubated with IgG control (Cell Signaling) or anti-YTHDF1/DF2/DF3/DC1/DC2 antibodies for 6 h to overnight at 4 °C, and then incubated with A/G Dynabeads for 2 hr at 4 °C. The beads were washed three times in detergent-free IP buffer with RNaseOUT (100 U/ml) and then incubated with proteinase K (30 □g, Ambion) in IP buffer (detergent-free, protease- and phosphatase inhibitor-free) containing 0.1% SDS. After 30 min digestion at 55 °C, Trizol was added to the beads and RNA isolated using RNeasy Mini kit (Qiagen) with on-column Dnase-I (Qiagen) digestion. Equal volumes of eluted RNA were used in one-step RT-qPCR with Ag-Path ID Kit. Fold-change in ΔΔCₜ values was obtained for EPRS1 mRNA with ACTB mRNA as control, from IP with YTHDF1/DF2/DF3/DC1/DC2 compared to IP with IgG for sibling LCLs. Similarly derived values from control LCLs were used as baseline to calculate fold-change of EPRS1 mRNA co-immunoprecipitated in RNP complexes with the m⁶A reader YTH proteins.

### Nucleofection of LCLs

For knockdown experiments, ~2 million control LCLs were nucleoporated with 300 nM targeting siRNA or Non-Targeting siRNA #1 (Silencer Select, Invitrogen), using program X05 on Nucleofector I or X005 on Nucleofector II, and Cell Line Nucleofector Kit V (Lonza), and cells collected after 72-75 hr. For experiments with nuclear TRM editors, ~2 million LCLs were nucleofected with a mix of 95% crRNA encoding pU6-PspCas13b plasmid and pCMV-dCas13b-METTL3-NLS or dCas13b-METTL3^{mut}-NLS plasmid at 1:2 molarity, and 5% pmaxGFP plasmid (Lonza) at a final amount of 2 mg plasmid DNA per nucleofection, using program X05 on Nucleofector I and Nucleofector Kit V (Lonza), and cells harvested after 96 h.

Evolutionary Conservation Analysis of G₄A:UC₄ Stem in EPRS1 mRNA

Gene ID for EPRS1 from multiple species were retrieved from NCBI Orthologs. Genomic sequences were aligned on Clustal Omega (Sievers et al., 2011). 24-nt windows, in-frame with the coding sequence, in the orthologous region surrounding the C4444A variant site (UC₄) arm and the opposite strand (G₄A) were curated. Frequency plots were generated on WebLogo (Crooks et al., 2004) and encoded amino acids annotated. Base-pair conservation in over 90% species were considered strong.

### RNA Structure Prediction

Mfold, RNAstructure, and RNAfold web server (ViennaRNA Package 2.0) energy minimization algorithms (Lorenz et al., 2011; Reuter and Mathews, 2010; Zuker, 2003) were used to fold RNA sequences.

TABLE 7
Eutheria (placental mammals)

| | | EPRS1 Gene |
|---|---|---|
| Common name | Scientfic name | ID (NCBI) |
| Human | Homo sapiens | 2058 |
| House mouse | Mus musculus | 107508 |
| Black rat | Rattus norvegicus | 289352 |
| Small eared galago | Otolemur garnettii | 100966256 |
| Ring-tailed lemur | Lemur catta | 123627005 |
| Gray mouse lemur | Microcebus murinus | 105886059 |
| Philippine tarsier | Carlito syrichta | 103257617 |
| Tufted capuchin | Sapajus apella | 116558954 |
| Rhesus monkey | Macaca mulatta | 706899 |
| Chimpanzee | Pan troglodytes | 457746 |
| Western lowland gorilla | Gorilla gorilla gorilla | 101132255 |
| Big brown bat | Eptesicus fuscus | 103293465 |
| Chinese tree shrew | Tupaia chinensis | 102472024 |
| Sunda flying lemur | Galeopterus variegatus | 103603328 |
| American beaver | Castor canadensis | 109682484 |
| Gray squirrel | Sciurus carolinensis | 124962055 |
| Rabbit | Oryctolagus cuniculus | 100340173 |
| Dog | Canis lupus familiaris | 478962 |
| Beluga whale | Delphinapterus leucas | 111177482 |
| Common bottlenose dolphin | Tursiops truncatus | 101332069 |
| Cow | Bos taurus | 538357 |
| Sheep | Ovis aries | 101104590 |
| Common vampire bat | Desmodus rotundus | 112318748 |

Reptilia

| | | EPRS1 Gene |
|---|---|---|
| Common name | Scientfic name | ID (NCBI) |
| Tiger rattlesnake | Crotalus tigris | 120307188 |
| Mainland tigersnake | Notechis scutatus | 113421898 |
| Western terrestrial garter | Thamnophis elegans | 116507382 |
| snake | | |
| Common garter snake | Thamnophis sirtalis | 106547182 |
| Chinese alligator | Alligator sinensis | 102381974 |
| American alligator | Alligator mississippiensis | 102565153 |
| Reevess turtle | Mauremys mutica | 120400723 |

Prediction of m⁶Ad-SNVs

The pipeline described here generates a comprehensive software solution implemented in Python (Van Rossum and Drake, 2009) for analysis of variant-dependent m⁶A modifications within the human genome. The software takes as input three distinct files: a FASTA file containing the human genome sequences, a VCF file containing a curated list of variations sourced from the ClinVar database (Landrum et al., 2016) intersected with RMVar (Luo et al., 2021) to focus on m⁶A modifications only, and a BED file specifying a series of genomic regions of interest. Human genome version GRCh38 was used, and the BED file contains genomic coordinates of protein-coding, whole gene regions with transcripts from NCBI RefSeq extracted from the UCSC Genome Browser (Lee et al., 2020).

Initially, the BED file is processed to extract, for each listed transcript, the genomic coordinates of the last two coding sequence exons, along with up to 100 base pairs of untranslated regions (UTRs) adjacent to the CDS. Once the BED file is processed, the pipeline iterates through the VCF entries with the m⁶A modifications. The occurrence of each modification in the BED file is verified across the regions described above. Upon a positive match, the nucleotide sequences of the corresponding CDS and UTR segments, per the relevant transcript, are retrieved from the input genome FASTA file, yielding a reference sequence. Subsequently, an alternate sequence is generated by applying the modification specified in the VCF entry. The pipeline proceeds by searching for DRACH sites within the reference sequence through a regular expression (DRACH→[AGT][AG]AC[ACT]). Modifications overlapping DRACH motifs, causing termination codons or frameshifts, or altering termination codons to sense codons, are rejected. Following these quality filters, we predict the secondary structure of the reference and alternate sequences using ViennaRNA RNAfold (Lorenz et al., 2011), with a single constraint that isolated base pairs are not formed.

The primary objective is to assess if m⁶A-distal SNVs affect DRACH site accessibility, specifically by evaluating the alteration of base-pairing of nucleotides within segments of the DRACH motif, i.e., DRA, RAC, ACH, DRAC, RACH, and the entire DRACH. m⁶A-site accessibility, as reflected by DRACH-site base-pairing, was used as a scoring system for ranking the identified targets. The evaluation compares the reference and alternate dot-bracket representation of the folding structures at the DRACH level, with non-overlapping scoring, e.g., in the case of a free DRAC site, the counter of free DRAC sites is incremented, but the same site is not considered for counting free DRA and RAC sites. The final pipeline output is a table that reports filtered m⁶A sites with altered availability, and relevant information, including ClinVar ID, genomic coordinates, strand orientation, associated gene symbol, NCBI RefSeq transcript ID, modification position, minimum free energy (MFE) for reference and alternate structures, ΔMFE (difference between the absolute values of MFEs), a flag reporting whether the modification is synonymous, the total number of DRACH sites in both the reference and alternate structures, and the number of free and paired DRA, RAC, ACH, DRAC, RACH, and DRACH sites in both the reference and alternate structures. The table of predicted m⁶Ad-SNV candidates is interactive and permits visual inspection of predicted structures rendered with the forma JavaScript library (Kerpedjiev et al., 2015), with the length of the reference and m⁶Ad-SNV-containing alternate sequences limited to 250 base pairs. Within this constraint, applied to maintain reliability of the results predicted by RNAfold, the sequence composition contains up to 100 base pairs from 3′UTRs, with the remaining base pairs limited to the last two exons.

### Widespread m⁶A Site-Distal Single-Nucleotide Variants Predicted to Alter DRACH Site Accessibility

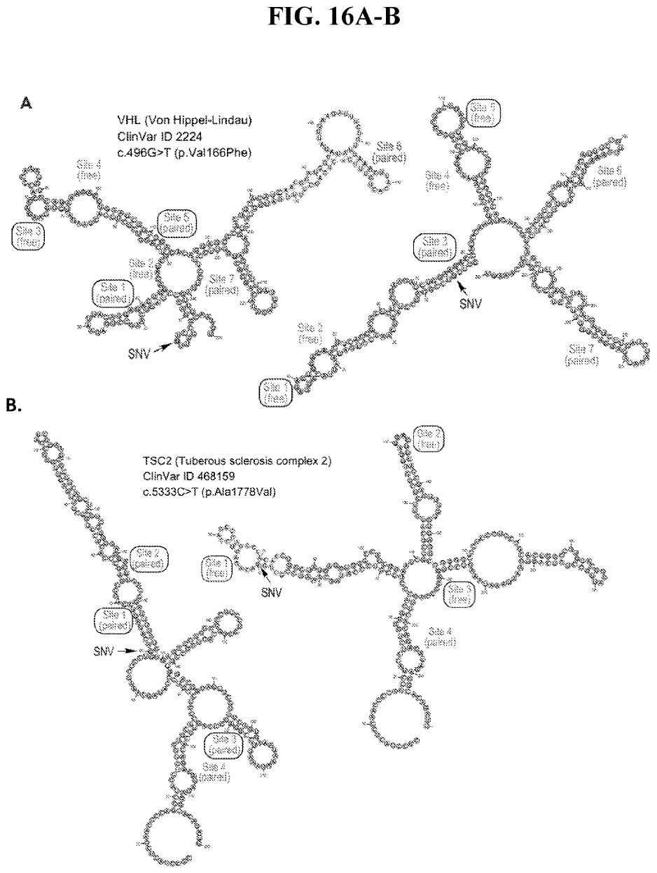

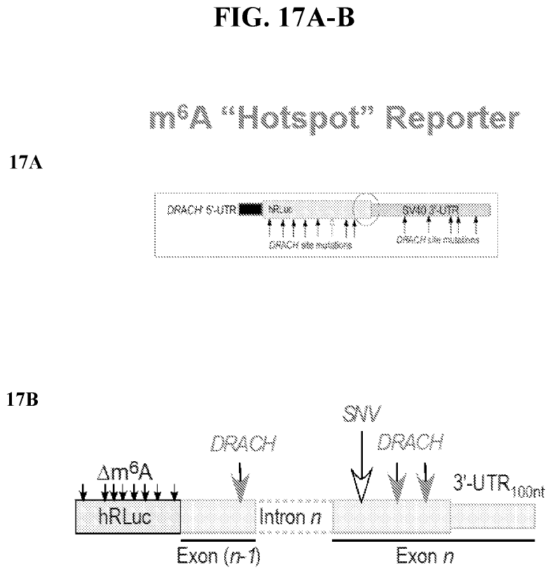

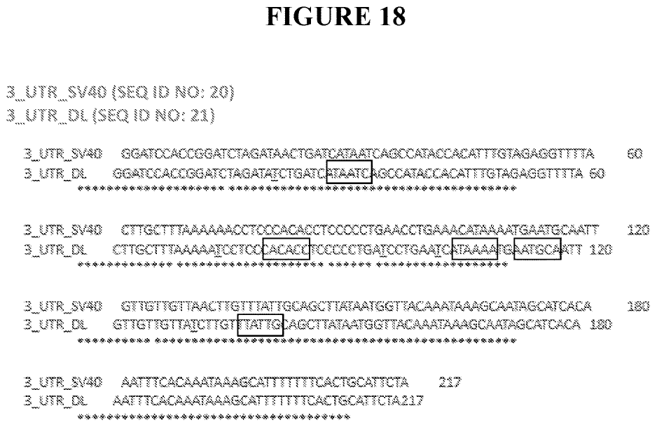

The prospect of additional single-nucleotide variants (SNV) that bury or expose distal DRACH sites through altered base-pairing was investigated. SNVs in the ClinVar database of health status-associated genomic variations was cross-referenced with validated m⁶A sites in the RMVar database of RNA base modifications for all NCBI Refseq transcripts. Analysis was confined to the neighborhood of the CDS-terminal “hotspot” region for m⁶A modifications (Dominissini et al., 2012), by using 100 nts of the 3′UTR following the stop-codon, and the preceding up to 150 nts sourced from up to the last two exons in the coding region. Energy-minimized, predicted secondary structures of the wild-type and ClinVar SNV-containing mRNAs were calculated with RNAfold, and changes in predicted base-pairing at DRACH sites were determined. 117 “hits” in 54 genes and 87 ClinVar SNVs were identified as candidate m⁶A-distal (m⁶Ad) SNVs (Table 3, Expanded Data). Notably, multiple ClinVar SNVs can alter m⁶A-site accessibility of a given gene, while multiple but not all transcripts of a gene can be affected by a single ClinVar SNV. Twenty candidates encompassing 11 genes and 14 ClinVar SNVs were from synonymous m⁶Ad-SNVs, potentially representing a new class of “silent” mutations that can alter gene expression and pathogenicity by altering m⁶A-site accessibility. DRACH-motif nucleotide base-pairing was scored as a measure of m⁶A site-accessibility (Meiser et al., 2020). As one example, a m⁶Ad-SNV in Von Hippel-Lindau mRNA (VHL, ClinVar ID 2224) is predicted to free two DRACH sites base-paired in the reference mRNA, and block accessibility of a third site (FIG. 16A). Similarly, a m⁶Ad-SNV in tuberous sclerosis complex 2 mRNA (TSC2, ClinVar ID 468159) predicts increased availability of three DRACH sites base-paired in the reference mRNA (FIG. 16B). These newly predicted m⁶Ad-SNVs, by altering accessibility of the methyltransferase to distant m⁶A sites, contrast with the established direct-acting m⁶A-SNPs that alter DRACH or near-DRACH sequences to inactivate existing m⁶A sites or generate new ones, respectively (Ruan et al., 2021). Importantly, as we have shown for the HLD-causing m⁶Ad-SNV in EPRS1 mRNA, pathologies induced by the newly revealed m⁶Ad-SNVs might be correctible by RNA-based therapeutics.

Widespread m6A site-distal single-nucleotide variants predicted to alter DRACH site accessibility. SNVs in the ClinVar database of health status-associated genomic variations were cross-referenced with validated m6A sites in the RMVar database of RNA base modifications for all NCBI Refseq transcripts. Analysis was confined to the neighborhood of the CDS-terminal hotspot region for m6A modifications by using 100 nucleotides of the 3′UTR following the stop-codon, and the preceding up to 150 nucleotides restricted to the last two exons in the coding region. Energy-minimized, predicted secondary structures of the wild-type and ClinVar SNV-containing mRNAs were calculated with RNAfold, and changes in predicted base-pairing at DRACH sites were determined. 117 hits in 54 genes and 87 ClinVar SNVs were identified as candidate m6A-distal (m6Ad) SNVs and are shown in Table 3.

TABLE 3
Predicted m⁶Ad-SNVs

| Gene Symbol | Transcript_ID | ClinVar ID | Gene Symbol | Transcript_ID | ClinVar ID |
|---|---|---|---|---|---|
| ABCG8 | NM_001357321.2 | 336092 | PALB2 | NM_001407309.1 | 141936 |
| AIP | NM_003977.4 | 485069 | PALB2 | NM_001407304.1 | 182776 |
| BBS10 | NM_024685.4 | 310487 | PALB2 | NM_001407309.1 | 182776 |
| BMPR1A | NM_001406566.1 | 486801 | PALB2 | NM_001407304.1 | 182777 |
| BRAT1 | NM_001350626.2 | 540173 | PALB2 | NM_001407309.1 | 182777 |
| BRAT1 | NM_152743.4 | 540173 | PALB2 | NM_001407310.1 | 229790 |
| BRCA2 | NM_001406722.1 | 409527 | PALB2 | NM_001407304.1 | 234009 |
| CACNB2 | NM_201597.3 | 136649 | PALB2 | NM_001407309.1 | 234009 |
| CACNB2 | NM_201597.3 | 698890 | PALB2 | NM_001407304.1 | 378321 |
| CBS | NM_001321072.1 | 413352 | PALB2 | NM_001407309.1 | 378321 |
| CBS | NM_001178009.3 | 413352 | PALB2 | NM_001407310.1 | 460993 |
| CDKN1B | NM_004064.5 | 404269 | PALB2 | NM_001407304.1 | 484208 |
| CDKN1B | NM_004064.5 | 536845 | PALB2 | NM_001407309.1 | 484208 |
| CFAP418 | NM_177965.4 | 31194 | PALB2 | NM_001407304.1 | 530116 |
| CFAP418 | NM_001363260.1 | 31194 | PALB2 | NM_001407310.1 | 530116 |
| CLN5 | NM_001366624.2 | 527741 | PALB2 | NM_001407309.1 | 530116 |
| CTNS | NM_001374496.1 | 322845 | PALB2 | NM_001407310.1 | 530122 |
| CTNS | NM_001031681.3 | 322846 | PALB2 | NM_001407304.1 | 628361 |
| DKC1 | NM_001142463.3 | 580161 | PALB2 | NM_001407309.1 | 628361 |
| DNAI1 | NM_012144.4 | 454834 | PLEKHG5 | NM_001265594.3 | 581116 |
| EARS2 | NM_001308211.1 | 318541 | PMS2 | NM_001406877.1 | 480318 |
| ENSG00000256591 | NM_017841.4 | 463828 | PMS2 | NM_001406875.1 | 480318 |
| ENSG00000267618 | NM_001142571.2 | 141707 | PMS2 | NM_001406871.1 | 480318 |
| ENSG00000267618 | NM_001142571.2 | 630954 | PRICKLE1 | NM_001144881.2 | 1291049 |
| ENSG00000273269 | NM_001305626.1 | 458192 | SDHB | NM_003000.3 | 412459 |
| FBN1 | NM_001406716.1 | 519710 | SIK1 | NM_173354.5 | 476102 |
| FGD4 | NM_001370298.3 | 543382 | SMAD3 | NM_001407012.1 | 213777 |
| FH | NM_000143.4 | 460345 | SMAD4 | NM_001407041.1 | 492457 |
| FHL1 | NM_001159702.3 | 11559 | SMAD4 | NM_001407041.1 | 630655 |
| FKTN | NM_001351498.2 | 36137 | SP110 | NM_080424.4 | 334890 |
| FKTN | NM_001198963.2 | 364489 | SP110 | NM_001378446.1 | 334890 |
| FKTN | NM_001351498.2 | 459218 | SP110 | NM_001378446.1 | 577709 |
| FKTN | NM_001198963.2 | 459218 | TGFBI | NM_000358.3 | 350898 |
| GLE1 | NM_001003722.2 | 6465 | THRB | NM_001252634.2 | 12564 |
| GNRHR | NM_001012763.2 | 349452 | THRB | NM_001374827.1 | 12564 |
| GUSB | NM_000181.4 | 92588 | TIMM8A | NM_004085.4 | 11321 |
| HNRNPA1 | NM_002136.4 | 135603 | TSC2 | NM_001406686.1 | 49370 |
| HYCC1 | NM_032581.4 | 359768 | TSC2 | NM_001370405.1 | 49370 |
| IDUA | NM_000203.5 | 100723 | TSC2 | NM_001406686.1 | 65302 |
| IFT74 | NM_001099223.3 | 254276 | TSC2 | NM_001370405.1 | 65302 |
| IRF2BP2 | NM_001077397.1 | 446216 | TSC2 | NM_001406686.1 | 468159 |
| IRF2BP2 | NM_182972.3 | 446216 | TSC2 | NM_001370405.1 | 468159 |
| KCTD7 | NM_153033.5 | 469108 | TSC2 | NM_001406686.1 | 486616 |
| KCTD7 | NM_001167961.2 | 469108 | TSC2 | NM_001370405.1 | 486616 |
| KIRREL3 | NM_032531.4 | 2888 | TSC2 | NM_001406686.1 | 535891 |
| MCL1 | NM_182763.3 | 623464 | TSC2 | NM_001370405.1 | 535891 |
| MEN1 | NM_001407150.1 | 305312 | TSC2 | NM_001406686.1 | 536004 |
| MEN1 | NM_001407152.1 | 305312 | TSC2 | NM_001370405.1 | 536004 |
| MSH2 | NM_001406648.1 | 90999 | TSC2 | NM_001406686.1 | 655006 |
| MSH2 | NM_001406648.1 | 91000 | TSC2 | NM_001370405.1 | 655006 |
| MSH2 | NM_001406648.1 | 387732 | TSC2 | NM_001406686.1 | 1006099 |
| MSH2 | NM_001406657.1 | 489918 | TSC2 | NM_001370405.1 | 1006099 |
| MSMO1 | NM_001017369.3 | 222976 | TSC2 | NM_001406686.1 | 1019286 |
| NR4A2 | NM_006186.4 | 331658 | TSC2 | NM_001370405.1 | 1019286 |
| PALB2 | NM_001407304.1 | 126741 | TXNRD2 | NM_001352303.2 | 264270 |
| PALB2 | NM_001407309.1 | 126741 | VCL | NM_014000.3 | 487634 |
| PALB2 | NM_001407304.1 | 126745 | VHL | NM_198156.3 | 2224 |
| PALB2 | NM_001407309.1 | 126745 | VHL | NM_000551.4 | 2224 |
| PALB2 | NM_001407304.1 | 141936 | | | |

### REFERENCES

Arif, A., Terenzi, F., Potdar, A. A., Jia, J., Sacks, J., China, A., Halawani, D., Vasu, K., Li, X., Brown, J. M., et al. (2017). EPRS is a critical mTORC1-S6K1 effector that influences adiposity in mice. Nature 542, 357-361.
Barbarese, E., Koppel, D. E., Deutscher, M. P., Smith, C. L., Ainger, K., Morgan, F., and Carson, J. H. (1995). Protein translation components are colocalized in granules in oligodendrocytes. J Cell Sci 108, 2781-2790.
Boespflug-Tanguy, O., Aubourg, P., Dorboz, T., Begou, M., Giraud, G., Sarret, C., and Vaurs-Barriere, C. (2011). Neurodegenerative disorder related to AIMP1/p43 mutation is not a PMLD. Am J Hum Genet 88, 392-393.
Boissel, S., Reish, O., Proulx, K., Kawagoe-Takaki, H., Sedgwick, B., Yeo, G. S., Meyre, D., Golzio, C., Molinari, F., Kadhom, N., et al. (2009). Loss-of-function mutation in the dioxygenase-encoding FTO gene causes severe growth retardation and multiple malformations. Am J Hum Genet 85, 106-111.
Carmody, S. R., and Wente, S. R. (2009). mRNA nuclear export at a glance. J Cell Sci 122, 1933-1937.
Chang, G., Shi, L., Ye, Y., Shi, H., Zeng, L., Tiwary, S., Huse, J. T., Huo, L., Ma, L., Ma, Y., et al. (2020). YTHDF3 induces the translation of m6A-enriched gene transcripts to promote breast cancer brain metastasis. Cancer Cell 38, 857-871 e857.
Choquet, K., Yang, S., Moir, R. D., Forget, D., Lariviere, R., Bouchard, A., Poitras, C., Sgarioto, N., Dicaire, M. J., Noohi, F., et al. (2017). Absence of neurological abnormalities in mice homozygous for the Polr3a G672E hypomyelinating leukodystrophy mutation. Mol Brain 10, 13.
Cui, H., Kapur, M., Diedrich, J. K., Yates, J. R., Ackerman, S. L., and Schimmel, P. (2021). Regulation of ex-translational activities is the primary function of the multi-tRNA synthetase complex. Nucleic Acids Res 49, 3603-3616.
David, A., Netzer, N., Strader, M. B., Das, S. R., Chen, C. Y., Gibbs, J., Pierre, P., Bennink, J. R., and Yewdell, J. W. (2011). RNA binding targets aminoacyl-tRNA synthetases to translating ribosomes. J Biol Chem 286, 20688-20700.
Dominissini, D., Moshitch-Moshkovitz, S., Schwartz, S., Salmon-Divon, M., Ungar, L., Osenberg, S., Cesarkas, K., Jacob-Hirsch, J., Amariglio, N., Kupiec, M., et al. (2012). Topology of the human and mouse m6A RNA methylomes revealed by m6A-seq. Nature 485, 201-206.
Du, H., Zhao, Y., He, J., Zhang, Y., Xi, H., Liu, M., Ma, J., and Wu, L. (2016). YTHDF2 destabilizes m6A-containing RNA through direct recruitment of the CCR4-NOT deadenylase complex. Nat Commun 7, 12626.
Elitt, M. S., Barbar, L., Shick, H. E., Powers, B. E., Maeno-Hikichi, Y., Madhavan, M., Allan, K. C., Nawash, B. S., Gevorgyan, A. S., Hung, S., et al. (2020). Suppression of proteolipid protein rescues Pelizaeus-Merzbacher disease. Nature 585, 397-403.
Elitt, M. S., Shick, H. E., Madhavan, M., Allan, K. C., Clayton, B. L. L., Weng, C., Miller, T. E., Factor, D. C., Barbar, L., Nawash, B. S., et al. (2018). Chemical screening identifies enhancers of mutant oligodendrocyte survival and unmasks a distinct pathological phase in Pelizaeus-Merzbacher Disease. Stem Cell Reports 11, 711-726. Feinstein, M., Markus, B., Noyman, I., Shalev, H., Flusser, H., Shelef, I., Liani-Leibson, K., Shorer, Z., Cohen, I., Khateeb, S., et al. (2010). Pelizaeus-Merzbacher-like disease caused by AIMP1/p43 homozygous mutation. Am J Hum Genet 87, 820-828.
Frank, D. E., Schnell, F. J., Akana, C., El-Husayni, S. H., Desjardins, C. A., Morgan, J., Charleston, J. S., Sardone, V., Domingos, J., Dickson, G., et al. (2020). Increased dystrophin production with golodirsen in patients with Duchenne muscular dystrophy. Neurology 94, e2270-e2282.
Halawani, D., Gogonea, V., DiDonato, J. A., Pipich, V., Yao, P., China, A., Topbas, C., Vasu, K., Arif, A., Hazen, S. L., et al. (2018). Structural control of caspase-generated glutamyl-tRNA synthetase by appended noncatalytic WHEP domains. J Biol Chem 293, 8843-8860.
Hull, V., Wang, Y., Burns, T., Zhang, S., Sternbach, S., McDonough, J., Guo, F., and Pleasure, D. (2020). Antisense oligonucleotide reverses leukodystrophy in Canavan disease mice. Ann Neurol 87, 480-485.
Hunt, R. C., Simhadri, V. L., Iandoli, M., Sauna, Z. E., and Kimchi-Sarfaty, C. (2014). Exposing synonymous mutations. Trends Genet 30, 308-321.
Itoh, M., Dai, H., Horike, S. I., Gonzalez, J., Kitami, Y., Meguro-Horike, M., Kuki, I., Shimakawa, S., Yoshinaga, H., Ota, Y., et al. (2019). Biallelic KARS pathogenic variants cause an early-onset progressive leukodystrophy. Brain 142, 560-573.
Jiang, X., Liu, B., Nie, Z., Duan, L., Xiong, Q., Jin, Z., Yang, C., and Chen, Y. (2021). The role of m6A modification in the biological functions and diseases. Signal Transduct Target Ther 6, 74.
Jin, D., Wek, S. A., Cordova, R. A., Wek, R. C., Lacombe, D., Michaud, V., and Musier-Forsyth, K. (2023). Aminoacylation-defective bi-allelic mutations in human EPRS1 associated with psychomotor developmental delay, epilepsy, and deafness. Clin Genet 103, 358-363.
Kaminska, M., Havrylenko, S., Decottignies, P., Le Marechal, P., Negrutskii, B., and Mirande, M. (2009). Dynamic organization of aminoacyl-tRNA synthetase complexes in the cytoplasm of human cells. J Biol Chem 284, 13746-13754.
Ke, S., Alemu, E. A., Mertens, C., Gantman, E. C., Fak, J. J., Mele, A., Haripal, B., Zucker-Scharff, I., Moore, M. J., Park, C. Y., et al. (2015). A majority of m6A residues are in the last exons, allowing the potential for 3′ UTR regulation. Genes Dev 29, 2037-2053.
Kretschmer, J., Rao, H., Hackert, P., Sloan, K. E., Hobartner, C., and Bohnsack, M. T. (2018). The m6A reader protein YTHDC2 interacts with the small ribosomal subunit and the 5′-3′ exoribonuclease XRN1. RNA 24, 1339-1350.
Kwon, N. H., Fox, P. L., and Kim, S. (2019). Aminoacyl-tRNA synthetases as therapeutic targets. Nat Rev Drug Discov 18, 629-650.
Lai, Y., Yue, Y., Liu, M., and Duan, D. (2006). Synthetic intron improves transduction efficiency of trans-splicing adeno-associated viral vectors. Hum Gene Ther 17, 1036-1042.
Lee, E. Y., Kim, S. M., Hwang, J. H., Jang, S. Y., Park, S., Choi, S., Lee, G. S., Hwang, J., Moon, J. H., Fox, P. L., et al. (2022). Glutamyl-prolyl-tRNA synthetase 1 coordinates early endosomal anti-inflammatory AKT signaling. Nat Commun 13, 6455.
Lee, E. Y., Lee, H. C., Kim, H. K., Jang, S. Y., Park, S. J., Kim, Y. H., Kim, J. H., Hwang, J., Kim, J. H., Kim, T. H., et al. (2016). Infection-specific phosphorylation of glutamyl-prolyl tRNA synthetase induces antiviral immunity. Nat Immunol 17, 1252-1262.
Lee, S. W., Cho, B. H., Park, S. G., and Kim, S. (2004). Aminoacyl-tRNA synthetase complexes: beyond translation. J Cell Sci 117, 3725-3734.
Lesbirel, S., Viphakone, N., Parker, M., Parker, J., Heath, C., Sudbery, I., and Wilson, S. A. (2018). The m6A-methylase complex recruits TREX and regulates mRNA export. Sci Rep 8, 13827.
Li, Z., Weng, H., Su, R., Weng, X., Zuo, Z., Li, C., Huang, H., Nachtergaele, S., Dong, L., Hu, C., et al. (2017). FTO plays an oncogenic role in acute myeloid leukemia as a N6-methyladenosine RNA demethylase. Cancer Cell 31, 127-141.
Liu, N., Dai, Q., Zheng, G., He, C., Parisien, M., and Pan, T. (2015). N6-methyladenosine-dependent RNA structural switches regulate RNA-protein interactions. Nature 518, 560-564.
Loening, A. M., Fenn, T. D., and Gambhir, S. S. (2007). Crystal structures of the luciferase and green fluorescent protein from Renilla reniformis. J Mol Biol 374, 1017-1028.
Mazaheri, M., Yavari, M., Zare Marzouni, H., Stufano, A., Lovreglio, P., S'Amore, S., and Jahantigh, H. R. (2022). Case report: Mutation in AIMP2/P38, the scaffold for the multi-tRNA synthetase complex, and association with progressive neurodevelopmental disorders. Front Genet 13, 816987.
McGlacken-Byrne, S. M., Del Valle, I., Quesne Stabej, P. L., Bellutti, L., Garcia-Alonso, L., Ocaka, L. A., Ishida, M., Suntharalingham, J. P., Gagunashvili, A., Ogunbiyi, O. K., et al. (2022). Pathogenic variants in the human m6A reader YTHDC2 are associated with primary ovarian insufficiency. JCI Insight 7, e154671.
McLaughlin, H. M., Sakaguchi, R., Liu, C., Igarashi, T., Pehlivan, D., Chu, K., Iyer, R., Cruz, P., Cherukuri, P. F., Hansen, N. F., et al. (2010). Compound heterozygosity for loss-of-function lysyl-tRNA synthetase mutations in a patient with peripheral neuropathy. Am J Hum Genet 87, 560-566.
McMillan, H. J., Humphreys, P., Smith, A., Schwartzentruber, J., Chakraborty, P., Bulman, D. E., Beaulieu, C. L., Consortium, F. C., Majewski, J., Boycott, K. M., et al. (2015). Congenital visual impairment and progressive microcephaly due to lysyl-transfer ribonucleic acid (RNA) synthetase (KARS) mutations: the expanding phenotype of aminoacyl-transfer RNA synthetase mutations in human disease. J Child Neurol 30, 1037-1043.
Meiser, N., Mench, N., and Hengesbach, M. (2020). RNA secondary structure dependence in METTL3-METTL14 mRNA methylation is modulated by the N-terminal domain of METTL3. Biol Chem 402, 89-98.
Mendell, J. R., Rodino-Klapac, L. R., Sahenk, Z., Roush, K., Bird, L., Lowes, L. P., Alfano, L., Gomez, A. M., Lewis, S., Kota, J., et al. (2013). Eteplirsen for the treatment of Duchenne muscular dystrophy. Ann Neurol 74, 637-647.
Mendes, M. I., Green, L. M. C., Bertini, E., Tonduti, D., Aiello, C., Smith, D., Salsano, E., Beerepoot, S., Hertecant, J., von Spiczak, S., et al. (2020). RARS1-related hypomyelinating leukodystrophy: Expanding the spectrum. Ann Clin Transl Neurol 7, 83-93.
Mendes, M. I., Gutierrez Salazar, M., Guerrero, K., Thiffault, I., Salomons, G. S., Gauquelin, L., Tran, L. T., Forget, D., Gauthier, M. S., Waisfisz, Q., et al. (2018). Bi-allelic mutations in EPRS, encoding the glutamyl-prolyl-aminoacyl-tRNA synthetase, cause a hypomyelinating leukodystrophy. Am J Hum Genet 102, 676-684.
Meyer, K. D., Saletore, Y., Zumbo, P., Elemento, O., Mason, C. E., and Jaffrey, S. R. (2012). Comprehensive analysis of mRNA methylation reveals enrichment in 3′ UTRs and near stop codons. Cell 149, 1635-1646.
Nafisinia, M., Sobreira, N., Riley, L., Gold, W., Uhlenberg, B., Weiss, C., Boehm, C., Prelog, K., Ouvrier, R., and Christodoulou, J. (2017). Mutations in RARS cause a hypomyelination disorder akin to Pelizaeus-Merzbacher disease. Eur J Hum Genet 25, 1134-1141.
Negrutskii, B. S., and Deutscher, M. P. (1991). Channeling of aminoacyl-tRNA for protein synthesis in vivo. Proc Natl Acad Sci USA 88, 4991-4995.
Netzer, N., Goodenbour, J. M., David, A., Dittmar, K. A., Jones, R. B., Schneider, J. R., Boone, D., Eves, E. M., Rosner, M. R., Gibbs, J. S., et al. (2009). Innate immune and chemically triggered oxidative stress modifies translational fidelity. Nature 462, 522-526.
Niu, C., and Zhou, R. (2022). m6A-SNP: From genetics to epigenetics (Review). Int J Epigenetics 2, 1.
Patil, D. P., Pickering, B. F., and Jaffrey, S. R. (2018). Reading m(6)A in the transcriptome: m(6)A-binding proteins. Trends Cell Biol 28, 113-127.
Pouwels, P. J., Vanderver, A., Bernard, G., Wolf, N. I., Dreha-Kulczewksi, S. F., Deoni, S. C., Bertini, E., Kohlschutter, A., Richardson, W., Ffrench-Constant, C., et al. (2014). Hypomyelinating leukodystrophies: translational research progress and prospects. Ann Neurol 76, 5-19.
Reuter, J. S., and Mathews, D. H. (2010). RNAstructure: software for RNA secondary structure prediction and analysis. BMC Bioinformatics 11, 129.
Roundtree, I. A., Luo, G. Z., Zhang, Z., Wang, X., Zhou, T., Cui, Y., Sha, J., Huang, X., Guerrero, L., Xie, P., et al. (2017). YTHDC1 mediates nuclear export of N6-methyladenosine methylated mRNAs. Elife 6, e31311.
Rutherford, H. A., and Hamilton, N. (2019). Animal models of leukodystrophy: a new perspective for the development of therapies. FEBS J 286, 4176-4191.
Sampath, P., Mazumder, B., Seshadri, V., Gerber, C. A., Chavatte, L., Kinter, M., Ting, S. M., Dignam, J. D., Kim, S., Driscoll, D. M., and Fox, P. L. (2004). Noncanonical function of glutamyl-prolyl-tRNA synthetase: gene-specific silencing of translation. Cell 119, 195-208.
Scotto-Lavino, E., Du, G., and Frohman, M. A. (2006). 3′ end cDNA amplification using classic RACE. Nat Protoc 1, 2742-2745.
Shi, H., Wang, X., Lu, Z., Zhao, B. S., Ma, H., Hsu, P. J., Liu, C., and He, C. (2017). YTHDF3 facilitates translation and decay of N6-methyladenosine-modified RNA. Cell Res 27, 315-328.
Shukla, A., Das Bhowmik, A., Hebbar, M., Rajagopal, K. V., Girisha, K. M., Gupta, N., and Dalal, A. (2018). Homozygosity for a nonsense variant in AIMP2 is associated with a progressive neurodevelopmental disorder with microcephaly, seizures, and spastic quadriparesis. J Hum Genet 63, 19-25.
Shulman, Z., and Stern-Ginossar, N. (2020). The RNA modification N6-methyladenosine as a novel regulator of the immune system. Nat Immunol 21, 501-512.
Spitale, R. C., Flynn, R. A., Zhang, Q. C., Crisalli, P., Lee, B., Jung, J. W., Kuchelmeister, H. Y., Batista, P. J., Torre, E. A., Kool, E. T., et al. (2015). Structural imprints in vivo decode RNA regulatory mechanisms. Nature 519, 486-490.
Sun, X., Wang, D. O., and Wang, J. (2022). Targeted manipulation of m6A RNA modification through CRISPR-Cas-based strategies. Methods 203, 56-61.
Taft, R. J., Vanderver, A., Leventer, R. J., Damiani, S. A., Simons, C., Grimmond, S. M., Miller, D., Schmidt, J., Lockhart, P. J., Pope, K., et al. (2013). Mutations in DARS cause hypomyelination with brain stem and spinal cord involvement and leg spasticity. Am J Hum Genet 92, 774-780.
Tang, Y., Chen, K., Song, B., Ma, J., Wu, X., Xu, Q., Wei, Z., Su, J., Liu, G., Rong, R., et al. (2021). m6A-Atlas: a comprehensive knowledgebase for unraveling the N6-methyladenosine (m6A) epitranscriptome. Nucleic Acids Res 49, D134-D143.
Tantzer, S., Sperle, K., Kenaley, K., Taube, J., and Hobson, G. M. (2018). Morpholino antisense oligomers as a potential therapeutic option for the correction of alternative splicing in PMD, SPG2, and HEMS. Mol Ther Nucleic Acids 12, 420-432.
Tian, J., Ying, P., Ke, J., Zhu, Y., Yang, Y., Gong, Y., Zou, D., Peng, X., Yang, N., Wang, X., et al. (2020). ANKLE1 N6-Methyladenosine-related variant is associated with colorectal cancer risk by maintaining the genomic stability. Int J Cancer 146, 3281-3293.
Uddin, M. B., Roy, K. R., Hosain, S. B., Khiste, S. K., Hill, R. A., Jois, S. D., Zhao, Y., Tackett, A. J., and Liu, Y. Y. (2019). An N6-methyladenosine at the transited codon 273 of p53 pre-mRNA promotes the expression of R273H mutant protein and drug resistance of cancer cells. Biochem Pharmacol 160, 134-145.
Valencia, P., Dias, A. P., and Reed, R. (2008). Splicing promotes rapid and efficient mRNA export in mammalian cells. Proc Natl Acad Sci USA 105, 3386-3391.
van der Knaap, M. S., and Bugiani, M. (2017). Leukodystrophies: a proposed classification system based on pathological changes and pathogenetic mechanisms. Acta Neuropathol 134, 351-382.
van der Knaap, M. S., Schiffmann, R., Mochel, F., and Wolf, N. I. (2019). Diagnosis, prognosis, and treatment of leukodystrophies. Lancet Neurol 18, 962-972.
Vasu, K., Ramachandiran, I., Terenzi, F., Khan, D., China, A., Khan, K., Chechi, A., Baleanu-Gogonea, C., Gogonea, V., and Fox, P. L. (2021). The zinc-binding domain of mammalian prolyl-tRNA synthetase is indispensable for catalytic activity and organism viability. iScience 24, 102215.
Wang, X., Zhao, B. S., Roundtree, I. A., Lu, Z., Han, D., Ma, H., Weng, X., Chen, K., Shi, H., and He, C. (2015). N(6)-methyladenosine modulates messenger RNA translation efficiency. Cell 161, 1388-1399.
Wickramasinghe, V. O., and Laskey, R. A. (2015). Control of mammalian gene expression by selective mRNA export. Nat Rev Mol Cell Biol 16, 431-442.
Wilson, C., Chen, P. J., Miao, Z., and Liu, D. R. (2020). Programmable m6A modification of cellular RNAs with a Cas13-directed methyltransferase. Nat Biotechnol 38, 1431-1440.
Wolf, N. I., Salomons, G. S., Rodenburg, R. J., Pouwels, P. J., Schieving, J. H., Derks, T. G., Fock, J. M., Rump, P., van Beek, D. M., van der Knaap, M. S., et al. (2014). Mutations in RARS cause hypomyelination. Ann Neurol 76, 134-139.
Yang, C., Hu, Y., Zhou, B., Bao, Y., Li, Z., Gong, C., Yang, H., Wang, S., and Xiao, Y. (2020). The role of m6A modification in physiology and disease. Cell Death Dis 11, 960.
Zaccara, S., Ries, R. J., and Jaffrey, S. R. (2019). Reading, writing and erasing mRNA methylation. Nat Rev Mol Cell Biol 20, 608-624.
Zhang, X., Ling, J., Barcia, G., Jing, L., Wu, J., Barry, B. J., Mochida, G. H., Hill, R. S., Weimer, J. M., Stein, Q., et al. (2014). Mutations in QARS, encoding glutaminyl-tRNA synthetase, cause progressive microcephaly, cerebral-cerebellar atrophy, and intractable seizures. Am J Hum Genet 94, 547-558.
Zhou, H., Sun, L., Yang, X. L., and Schimmel, P. (2013). ATP-directed capture of bioactive herbal-based medicine on human tRNA synthetase. Nature 494, 121-124.

All publications and patents mentioned in the present application are herein incorporated by reference. Various modification and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method comprising:
a) testing a sample from a human subject and determining:
i) if said subject is A/A, A/C, or C/C at position 4444 in the Glutamyl-prolyl-tRNA synthetase 1 (EPRS1) gene or mRNA, and/or
ii) if said subject expresses only the 1482T version, only the 1482P version, or both the 1482T and 1482P versions, of the EPRS1 protein; and/or
iii) if said subject is m6A methylated or m6A unmethylated at A4355 and/or A4464 in said EPRS1 mRNA; and
b) generating, transmitting, and/or graphically displaying a report that:
i) said subject: A) is A/A at position 4444 in the EPRS1 gene or mRNA; and/or B) expresses only the 1482T version of EPRS1 protein; and/or C) is m6A unmethylated at A4355 and A4464 in said EPRS1 mRNA, and therefore said subject has a neurological disease and/or should be treated with a neurological therapeutic, nutritional therapy, hormone therapy, and/or physical, occupation, and/or speech therapy; and/or
ii) said subject: A) is A/C or C/C at position 4444 in the EPRS1 gene or mRNA; and/or B) expresses the 1482P version of EPRS1 protein; and/or C) is m6A methylated at A4355 and A4464 in said EPRS1 mRNA, and that said subject does not have hypomyelinating leukodystrophy (HLD).

2. The method of claim 1, wherein said neurological therapeutic comprises an HLD therapeutic, and/or wherein said nutritional therapy comprises HLD nutritional therapy, and/or wherein said hormone therapy comprises HLD hormone therapy, and/or wherein said physical, occupation, and/or speech therapy comprises HLD specific physical, occupation, and/or speech therapy, and/or wherein said indicates said subject has HLD.

3. The method of claim 1, wherein said neurological therapeutic is selected from:
a) a medication for seizures, muscle tightness and/or movement problems;
b) an oligonucleotide sequence that at least partially hybridizes to said EPRS1 mRNA changing the conformation of said EPRS1 mRNA thereby allowing m6A methylation at positions A4355 and A4464 of said EPRS1 mRNA to occur,
optionally wherein said oligonucleotide at least partially hybridizes between positions 4436 and 4464, or between positions 4327 and 4364;
c) a Cas targeting system comprising:
i) single guide RNA (sgRNA) comprising a targeting sequence (crRNA sequence) and a Cas nuclease-recruiting sequence (tracrRNA), and
ii) a fusion protein, or a expression vector encoding said fusion protein, wherein said fusion protein comprises: i) a catalytically-dead Cas enzyme, ii) a nuclear localization signal, and iii) at least a catalytically active portion of a methyltransferase that m6A methylates adenine.
wherein said Cas targeting system targets position A4355 and/or A4464 in said EPRS1 mRNA for methylation.

4. The method of claim 3, wherein said catalytically-dead Cas enzyme comprises a catalytically-dead Cas13b (dCas13b).

5. The method of claim 3, wherein said oligonucleotide sequence comprises a Phosphorodiamidate morpholino oligomer (PMO).

6. The method of claim 5, wherein said PMO is selected from the following: PMO2; PMO3; PMO4; PMO5; and PMO6.

7. The method of claim 1, wherein said subject is determined to be A/A at position 4444 in the EPRS1 gene or mRNA.

8. The method of claim 1, wherein said subject is determined to only express the 1482T version of EPRS1 protein.

9. The method of claim 1, wherein said subject is determined to be m6A unmethylated at A4355 and A4464 in said EPRS1 mRNA.

10. The method of claim 1, wherein said sample comprises a sample type selected from: saliva, serum, plasma, tissue biopsy, and whole blood.

11. A method comprising:
a) receiving and/or reviewing a report that a subject: i) is A/A at position 4444 in the EPRS1 gene or mRNA; and/or ii) expresses only the 1482T version of EPRS1 protein; and/or iii) is m6A unmethylated at A4355 and A4464 in said EPRS1 mRNA;
b) treating said subject with a neurological therapeutic, nutritional therapy, hormone therapy, and/or physical, occupation, and/or speech therapy.

12. The method of claim 11, wherein said neurological therapeutic comprises an HLD therapeutic, and/or wherein said nutritional therapy comprises HLD nutritional therapy, and/or wherein said hormone therapy comprises HLD hormone therapy, and/or wherein said physical, occupation, and/or speech therapy comprises HLD specific physical, occupation, and/or speech therapy, and/or wherein said indicates said subject has HLD.

13. The method of claim 11, wherein said neurological therapeutic is selected from:
a) a medication for seizures, muscle tightness and/or movement problems;
b) an oligonucleotide sequence that at least partially hybridizes to said EPRS1 mRNA changing the conformation of said EPRS1 mRNA thereby allowing m6A methylation at positions A4355 and A4464 of said EPRS1 mRNA to occur,
optionally wherein said oligonucleotide at least partially hybridizes between positions 4436 and 4464, or between positions 4327 and 4364;
c) a Cas targeting system comprising:
i) single guide RNA (sgRNA) comprising a targeting sequence (crRNA sequence) and a Cas nuclease-recruiting sequence (tracrRNA), and
ii) a fusion protein, or a expression vector encoding said fusion protein, wherein said fusion protein comprises: i) a catalytically-dead Cas enzyme, ii) a nuclear localization signal, and iii) at least a catalytically active portion of a methyltransferase that m6A methylates adenine;
wherein said Cas targeting system targets position A4355 and/or A4464 in said EPRS1 mRNA for methylation.

14. The method of claim 13, wherein said catalytically-dead Cas enzyme comprises a catalytically-dead Cas13b (dCas13b).

15. The method of claim 13, wherein said oligonucleotide sequence comprises a Phosphorodiamidate morpholino oligomer (PMO).

16. The method of claim 15, wherein said PMO is selected from the following:
PMO2; PMO3; PMO4; PMO5; and PMO6.

17. The method of claim 11, wherein said subject is determined to be A/A at position 4444 in the EPRS1 gene or mRNA.

18. The method of claim 11, wherein said subject is determined to only express the 1482T version of EPRS1 protein.

19. The method of claim 11, wherein said subject is determined to be m6A unmethylated at A4355 and A4464 in said EPRS1 mRNA.

20. A method of treating a subject with hypomyelinating leukodystrophy (HLD) comprising: administering any of the following to said subject:
a) an oligonucleotide sequence that at least partially hybridizes to EPRS1 mRNA changing the conformation of said EPRS1 mRNA thereby allowing m6A methylation at positions A4355 and A4464 of said EPRS1 mRNA to occur,
optionally wherein said oligonucleotide at least partially hybridizes between positions 4436 and 4464, or between positions 4327 and 4364; and/or
b) a Cas targeting system comprising:
i) single guide RNA (sgRNA) comprising a targeting sequence (crRNA sequence) and a Cas nuclease-recruiting sequence (tracrRNA), and
ii) a fusion protein, or a expression vector encoding said fusion protein, wherein said fusion protein comprises: i) a catalytically-dead Cas enzyme, ii) a nuclear localization signal, and iii) at least a catalytically active portion of a methyltransferase that m6A methylates adenine;
wherein said Cas targeting system targets position A4355 and/or A4464 in said EPRS1 mRNA for methylation.

21. The method of claim 20, wherein said catalytically-dead Cas enzyme comprises a catalytically-dead Cas13b (dCas13b).

22. The method of claim 20, wherein said oligonucleotide sequence comprises a Phosphorodiamidate morpholino oligomer (PMO).

23. The method of claim 22, wherein said PMO is selected from the following:
PMO2; PMO3; PMO4; PMO5; and PMO6.

24. A composition, kit, or system comprising:
a) an oligonucleotide sequence that at least partially hybridizes to EPRS1 mRNA changing the conformation of said EPRS1 mRNA thereby allowing m6A methylation at positions A4355 and A4464 of said EPRS1 mRNA to occur,
wherein said oligonucleotide comprises modified bases that reduce immunogenicity and/or wherein said oligonucleotide is a phosphorodiamidate morpholino oligomer (PMO);
optionally wherein said oligonucleotide at least partially hybridizes between positions 4436 and 4464, or between positions 4327 and 4364; and/or
b) a Cas targeting system comprising:
i) single guide RNA (sgRNA) comprising a targeting sequence (crRNA sequence) and a Cas nuclease-recruiting sequence (tracrRNA), and
ii) a fusion protein, or a expression vector encoding said fusion protein, wherein said fusion protein comprises: i) a catalytically-dead Cas enzyme, ii) a nuclear localization signal, and iii) at least a catalytically active portion of a methyltransferase that m6A methylates adenine; and
wherein said Cas targeting system targets position A4355 and/or A4464 in said EPRS1 mRNA for methylation.

25. The composition, kit, or system of claim 24, wherein said catalytically-dead Cas enzyme comprises a catalytically-dead Cas13b (dCas13b).

26. The composition, kit, or system of claim 24, wherein said oligonucleotide sequence comprises a Phosphorodiamidate morpholino oligomer (PMO).

27. The composition, kit, or system of claim 26, wherein said PMO is selected from the following: PMO2; PMO3; PMO4; PMO5; and PMO6.

28. The composition, kit, or system of claim 24, wherein said modified base is selected from: 5-methylcytidine (m5C), pseudouridine, and N1-Methylpseudouridine.

29. A composition comprising a nucleic acid reporter construct, wherein said nucleic acid reporter construct comprises:
a) a reporter sequence that generates a detectable signal when expressed in a cell, wherein said reporter sequence is free, or substantially free, of DRACH m6A methylation sites, and
b) a nucleic acid sequence of interest with a SNP site, wherein said nucleic acid sequence of interest is operably linked to said reporter sequence and comprises at least one DRACH site, and
wherein said at least one DRACH site:
i) does not contain said SNP site, and
ii) changes its m6A methylation status, but not its sequence, based on a change in the identity of a nucleotide at said SNP site.

30. The composition of claim 29, wherein said reporter construct further comprises an expression vector, and wherein said reporter sequence and nucleic acid sequence of interest are present in said expression vector.

31. The composition of claim 29, wherein said reporter sequence comprises at least a portion of a reporter gene modified to eliminate all, or substantially all, of DRACH sites present.

32. The composition of claim 31, wherein said reporter gene is selected from: beta-galactosidase, luciferase, beta-lactamase, alkaline phosphatase, and green fluorescence protein.

33. The composition of claim 29, wherein said a nucleic acid sequence of interest comprises at least a portion of a target gene selected from those recited in Table 3.

34. The composition of claim 33, wherein said SNP of said target gene is selected from those recited in Table 3.

35. The composition of claim 29, wherein said reporter sequence contains zero or one DRACH sites.

36. The composition of claim 29, wherein said nucleic acid sequence of interest comprises at least two DRACH sites, or at least three DRACH sites.

37. The composition of claim 29, wherein said identity of said nucleotide at said SNP site is the minor allele.

38. The composition of claim 29, wherein said identity of said nucleotide at said SNP site is known, or suspected of, being linked to a disease in a subject, wherein said subject is optionally a human.

39. The composition of claim 29, wherein said a nucleic acid sequence of interest comprises at least a portion of a target gene, wherein said at least a portion of a target gene comprise a terminal exon of said gene.

40. The composition of claim 29, wherein said a nucleic acid sequence of interest comprises at least a portion of a target gene, wherein said at least a portion of a target gene comprise the two terminal exons of said gene, and optionally an intron from said gene.

41. The composition of claim 29, wherein said nucleic acid construct further comprises a 5′ UTR free of DRACH sites.

42. The composition of claim 29, wherein said nucleic acid construct further comprises a 3′ UTR free of DRACH sites.

43. The composition of claim 31, wherein said 3′ UTR free of DRACH sites comprises an SV40 3′ UTR sequence free of DRACH sites.

44. The composition of claim 29, wherein said change its m6A methylation status is from methylated to non-methylated.

45. The composition of claim 29, wherein said change its m6A methylation status is from non-methylated to methylated.

46. A method comprising:
a) transfecting a cell with said nucleic acid construct of any of claims 29-46 where said SNP site has a minor allele or disease-causing allele, and detecting a first signal from said reporter sequence, and
b) transfecting a cell with said nucleic acid construct of any of claims 29-46 where said SNP site has a major allele or wild-type non-disease-causing allele, and detecting a second signal from said reporter sequence, and
c) comparing said first and second signals, optionally wherein said comparing comprises graphically comparing.

47. The method of claim 46, further comprising:
d) identifying said at least one DRACH site in said nucleic acid sequence of interest as having different m6A methylation when said minor allele or disease causing allele is present at said SNP site versus when said major allele or wild-type non-disease causing allele is present at said SNP site.

48. The method of claim 47, wherein said different m6A methylation identified indicates said SNP site changes methylation at said one or more DRACH sites in said nucleic acid sequence of interest.

49. A kit or system comprising:
a) a first nucleic acid construct of any of claims 29-46 where said SNP site has a minor allele or disease-causing allele, and
b) a second nucleic acid construct of any of claims 29-46 where said SNP site has a major allele or wild-type non-disease-causing allele.


SEQUENCE LISTING (21 sequences)

SEQ ID NO: 1 - DNA, 66 bp, Synthetic construct
  source 1..66: mol_type = other DNA
tggtttgaaggtgatgcaaaggcttttagcgttgtggaaggtccagttttgaggggctattacaac

SEQ ID NO: 2 - DNA, 66 bp, Synthetic construct
  source 1..66: mol_type = other DNA
gaaggtgatgcaaaggcttttagctcccatgttgtggaaggtccagttttgaggggctattacaac

SEQ ID NO: 3 - DNA, 66 bp, Synthetic construct
  source 1..66: mol_type = other DNA
tggtttgaaggggatgcaaaggcttttagcgttgtggaaggtccagttttgaggggctattacaac

SEQ ID NO: 4 - DNA, 66 bp, Synthetic construct
  source 1..66: mol_type = other DNA
gaaggggatgcaaaggcttttagctcccatgttgtggaaggtccagttttgaggggctattacaac

SEQ ID NO: 5 - DNA, 66 bp, Synthetic construct
  source 1..66: mol_type = other DNA
ccacagaatggaatctgaacaatctttccagttgtggaaggtccagttttgaggggctattacaac

SEQ ID NO: 6 - DNA, 66 bp, Synthetic construct
  source 1..66: mol_type = other DNA
aatggaatctgaacaatctttccagaatctgttgtggaaggtccagttttgaggggctattacaac

SEQ ID NO: 7 - DNA, 66 bp, Synthetic construct
  source 1..66: mol_type = other DNA
gcataagaataattgtcctgtgtgacttcagttgtggaaggtccagttttgaggggctattacaac

SEQ ID NO: 8 - DNA, 66 bp, Synthetic construct
  source 1..66: mol_type = other DNA
gaataattgtcctgtgtgacttcattttaggttgtggaaggtccagttttgaggggctattacaac

SEQ ID NO: 9 - DNA, 25 bp, Synthetic construct
  source 1..25: mol_type = other DNA
atttccccacagaatggaatctgaa

SEQ ID NO: 10 - DNA, 25 bp, Synthetic construct
  source 1..25: mol_type = other DNA
caatttccccacagaatggaatctg

SEQ ID NO: 11 - DNA, 25 bp, Synthetic construct
  source 1..25: mol_type = other DNA
tcacagagtggtttgaaggtgatgc

SEQ ID NO: 12 - DNA, 25 bp, Synthetic construct
  source 1..25: mol_type = other DNA
acagagtggtttgaaggtgatgcaa

SEQ ID NO: 13 - DNA, 25 bp, Synthetic construct
  source 1..25: mol_type = other DNA
cctcactccccacagaatggaatct

SEQ ID NO: 14 - DNA, 52 bp, Synthetic construct
  source 1..52: mol_type = other DNA
ccagtgagcagagtgacgaggactcgagctcaagcttttttttttttttttt

SEQ ID NO: 15 - DNA, 18 bp, Synthetic construct
  source 1..18: mol_type = other DNA
ccagtgagcagagtgacg

SEQ ID NO: 16 - DNA, 18 bp, Synthetic construct
  source 1..18: mol_type = other DNA
gaggactcgagctcaagc

SEQ ID NO: 17 - protein, 1344 aa, Synthetic construct
MKRTADGSEFESPKKKRKVNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDGSKRTADGSEFEPKKKRKVSGSETPGTSESATPESQEFCDYGTKEECMKASDADRPCRKLHFRRIINKHTDESLGDCSFLNTCFHMDTCKYVHYEIDACMDSEAPGSKDHTPSQELALTQSVGGDSSADRLFPPQWICCDIRYLDVSILGKFAVVMADPPWDIHMELPYGTLTDDEMRRLNIPVLQDDGFLFLWVTGRAMELGRECLNLWGYERVDEIIWVKTNQLQRIIRTGRTGHWLNHGKEHCLVGVKGNPQGFNQGLDCDVIVAEVRSTSHKPDEIYGMIERLSPGTRKIELFGRPHNVQPNWITLGNQLDGIHLLDPDVVARFKQRYPDGIISKPKNL

SEQ ID NO: 18 - DNA, 933 bp, Renilla sp.
  source 1..933: mol_type = genomic DNA
atggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcag

SEQ ID NO: 19 - DNA, 933 bp, Synthetic construct
  source 1..933: mol_type = other DNA
atggcttccaaggtgtacgaccccgagcaacgcaaacgcatgatcactgggcctcagtggtgggctcgctgcaagcaaatgaacgtgctcgattccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctcaatcttccaaagaaaatcatctttgtgggccacgactggggggcttgtctggcctttcactactcctacgagcaccaagataagatcaaggccatcgtccatgctgagagtgtcgtggacgtgatcgagtcctgggacgagtggcccgatatcgaggaggatatcgccctgatcaagagcgaagagggcgagaaaatggtgcttgagaataatttcttcgtcgagtccatgctcccaagcaagatcatgcggaagctggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccccaataccgagttcgtgaaggtgaagggcctccacttcagccaggaggacgctccagatgaaatgggtaagtacatcaagagcttcgtggagcgcgtgctgaagaacgagcag

SEQ ID NO: 20 - DNA, 217 bp, Simian virus 40
  source 1..217: mol_type = genomic DNA
ggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattcta

SEQ ID NO: 21 - DNA, 217 bp, Synthetic construct
  source 1..217: mol_type = other DNA
ggatccaccggatctagatatctgatcataatcagccataccacatttgtagaggttttacttgctttaaaaatcctcccacacctccccctgatcctgaatcataaaatgaatgcaattgttgttgttatcttgtttattgcagcttataatggttacaaataaagcaatagcatcacaaatttcacaaataaagcatttttttcactgcattcta